(19) United States
(12) Patent Application Publication
(10) Pub. No.: US 2024/0265534 A1
(43) Pub. Date: Aug. 8, 2024

(54) SYSTEMS AND DEVICES FOR AUTOMATED CELLULAR PROFILING AND METHODS OF USE THEREOF

(71) Applicant: CELLCHORUS INC., Houston, TX (US)

(72) Inventors: Navin Varadarajan, Houston, TX (US); Daniel D. Meyer, San Mateo, CA (US); Mohsen Fathi, Houston, TX (US)

(73) Assignee: CELLCHORUS INC., Houston, TX (US)

(21) Appl. No.: 18/565,482

(22) PCT Filed: Jun. 3, 2022
(86) PCT No.: PCT/US2022/032207

§ 371 Date: Nov. 29, 2023

Related U.S. Application Data
(60) Provisional application No. 63/337,252, filed on May 2, 2022.
(60) Provisional application No. 63/196,415, filed on Jun. 3, 2021.

(51) Int. Cl. G06T 7/00; C12M 1/12; C12M 1/34; G01N 33/50; G06T 7/70; G06V 20/69

(52) U.S. Cl. CPC G06T 7/0012; C12M 25/06; C12M 41/46; G01N 33/5091; G06T 7/70; G06V 20/69; G06T 2207/30024; G06T 2207/30072; G06T 2207/30242

(57) ABSTRACT

Systems and methods of present disclosure enable functional and molecular profiling with an incubation chamber that receives a well array and an imaging device in the incubation chamber. The well array has a sample of cells in at least one well. The imaging device captures a series of images of the filled well array during incubation in the incubation chamber and communicates the series of images to a processor. The processor detects each well in each image, localizes each well in each image, detects cells of the sample of cells in each well of each image, determines a number of cells in each well in each image based on the prioritization of the wells, tracks a relative location of each cell in each well, and determines cell-to-cell contact based on the relative location.

### CROSS-REFERENCE TO RELATED APPLICATIONS

This application claims priority to U.S. Provisional Application 63/337,252 filed on May 2, 2022, and to U.S. Provisional Application 63/196,415 filed on Jun. 3, 2021.

### FIELD OF TECHNOLOGY

The present disclosure generally relates to systems and devices configured for automated cellular profiling using cell imaging and methods of use thereof.

### BACKGROUND OF TECHNOLOGY

Immunotherapies have emerged as effective therapies for the treatment in oncology and other disease areas. The United States Food and Drug Administration has approved more than 30 immunotherapies for patients with cancers including bladder cancer, kidney cancer, leukemia, lung cancer, lymphoma, melanoma, and prostate cancer. Immunotherapies in therapeutic areas beyond oncology have also benefited from advances in immunotherapy. For example, significant advances have been made in addressing infectious diseases in recent years with vaccines, monoclonal-antibodies, T cell therapies and checkpoint inhibiters. Similarly, immunotherapy is showing potential to improve our understanding of diabetes, and to develop more specific treatments. While analysis modalities have been developed to characterize populations of T cells and other cells, these modalities cannot quantify motility and therapeutic potential of individual cells. The emergence of immunotherapies and other novel therapies requires modalities that can characterize statistically large populations of cells on an individual cell basis.

Existing modalities for quantifying the function of T cells and other cells are inadequate. While there have been significant advances in technologies that characterize single cells at the molecular level, these technologies are sample destructive and/or focused on information derived from nucleic acids (e.g., genomic, epigenomic, transcriptomic information). These modalities cannot characterize dynamic cell biology at the cellular or subcellular organelle resolution, and do not preserve cell viability for further downstream profiling.

Flow-cytometry is able to characterize the phenotype and cytokines of cells, but cannot characterize single cell dynamics and cell-cell interactions. Similarly, mass cytometry-based approaches together with barcoded antibodies are able to profiling subsets of cells, but do not maintain cell viability.

While many advances have been made in single-cell RNA sequencing (scRNA-seq), the approaches are sample destructive and cannot provide information on the motility, interactions and other functional performance of cells over time.

### SUMMARY OF DESCRIBED SUBJECT MATTER

Systems and methods of the present disclosure provide solutions for evaluating different cells such as CAR designs for CAR T and other cell therapies for applications such as determining which candidates to move forward in development. In some embodiments, the systems and methods characterize and/or predict response based on the functional performance (and possibly integrating other molecular profiling) of allogeneic immune cell products, antibodies, vaccine-exposed cells, target cell lines and/or patient-derived immune cells, disease cells and other cells, including before manufacturing, after manufacturing or after treatment. In some embodiments, the systems and methods evaluate the performance of manufactured products, to perform “release testing” or to monitor the consistency of manufacturing.

In some embodiments, the present disclosure provides solutions to enable the evaluation of dynamic and/or functional performance of cells and cell-cell interactions, as well as the ability to enable the direct link of dynamic/functional and molecular profiles of individual cells at high throughput and in a distributed fashion. The devices also enable dynamic imaging of mitochondria and function of individual T cells and other cells. The devices also allow for automated and user-assisted image processing. In some embodiments, the solutions may include cell loading that allows for desired distributions and ratios of one or more types of cells, which may be effector and/or target cells, for example a desired effector to target (E:T) ratio or effector to effector to target (E:E:T) ratio. The solutions may include devices containing arrays of nanowells that enable imaging cells and other inputs. The solutions may include loading that provides optimal distribution of capture beads and other detection elements. Accordingly, embodiments of the present disclosure provide improvements of a high throughput evaluation that may be performed using optimized methods for cell loading, faster image acquisition, image analysis and processing, improvement of cell labeling, improvement in label-free cell detection, etc.

### BRIEF DESCRIPTION OF THE DRAWINGS

Various embodiments of the present disclosure can be further explained with reference to the attached drawings, wherein like structures are referred to by like numerals throughout the several views. The drawings shown are not necessarily to scale, with emphasis instead generally being placed upon illustrating the principles of the present disclosure. Therefore, specific structural and functional details disclosed herein are not to be interpreted as limiting, but merely as a representative basis for teaching one skilled in the art to variously employ one or more illustrative embodiments.

FIG. 1 is a block diagram of a system for functional and molecular cell profiling in accordance with one or more embodiments of the present disclosure.

FIG. 2 illustrates a flowchart of an illustrative methodology cell preparation for functional and molecular cell profiling in accordance with one or more embodiments of the present disclosure.

FIG. 3 illustrates a flowchart of an illustrative methodology cassette loading for functional and molecular cell profiling in accordance with one or more embodiments of the present disclosure.

FIG. 4 illustrates a flowchart of an illustrative methodology for cassette preparation for functional and molecular cell profiling in accordance with one or more embodiments of the present disclosure.

FIG. 5 illustrates a flowchart of an illustrative methodology cell/bead loading into a cassette for functional and molecular cell profiling in accordance with one or more embodiments of the present disclosure.

FIG. 6A is a block diagram of an image analysis system for functional and molecular cell profiling in accordance with one or more embodiments of the present disclosure.

FIG. 6B illustrates a flowchart of an illustrative methodology for image analysis for functional and molecular cell profiling in accordance with one or more embodiments of the present disclosure.

FIG. 6C illustrates a flowchart of another illustrative methodology for image analysis for functional and molecular cell profiling in accordance with one or more embodiments of the present disclosure.

FIG. 7 depicts a block diagram of an exemplary computer-based system and platform for functional and molecular cell profiling in accordance with one or more embodiments of the present disclosure.

FIG. 8 depicts a block diagram of another exemplary computer-based system and platform for functional and molecular cell profiling in accordance with one or more embodiments of the present disclosure.

FIG. 9 depicts illustrative schematics of an exemplary implementation of the cloud computing/architecture(s) in which embodiments of a system for functional and molecular cell profiling may be specifically configured to operate in accordance with some embodiments of the present disclosure.

FIG. 10 depicts illustrative schematics of another exemplary implementation of the cloud computing/architecture(s) in which embodiments of a system for functional and molecular cell profiling may be specifically configured to operate in accordance with some embodiments of the present disclosure.

FIG. 11A illustrates a nanowell profiler window displays a table of single-cell measurements with each row linked to a specific nanowell video in accordance with aspects of embodiments of the present invention.

FIG. 111B, illustrates video display and editing window for the selected nanowell showing the contents of this nanowell with segmentation masks overlaid in accordance with aspects of embodiments of the present invention.

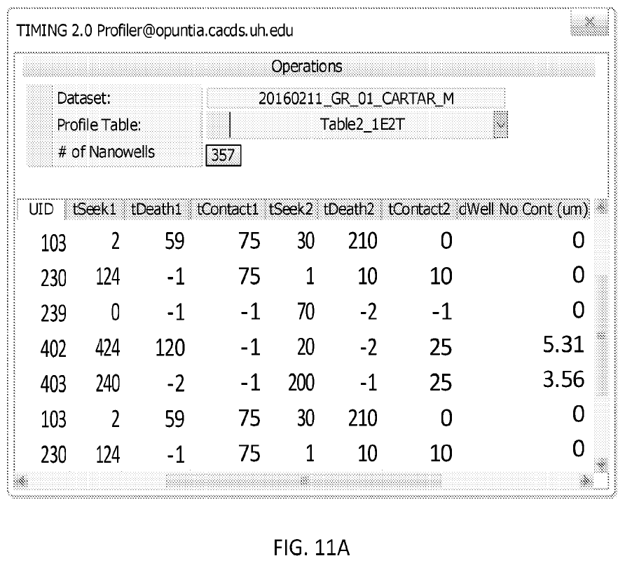

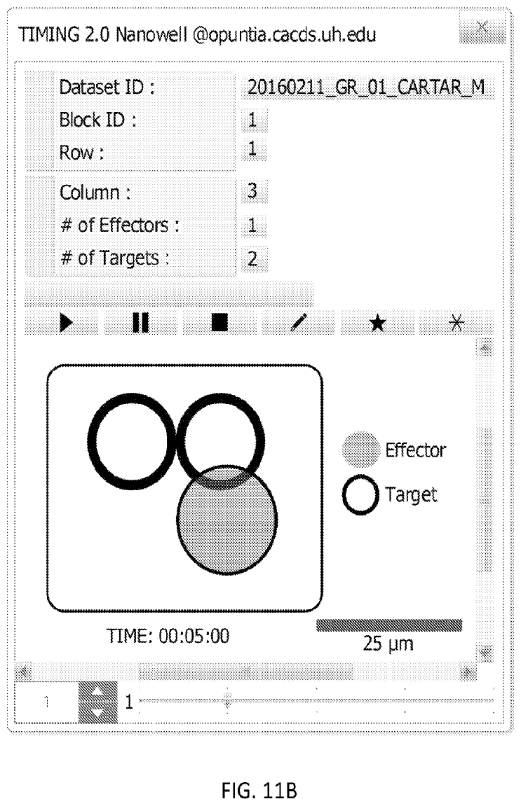

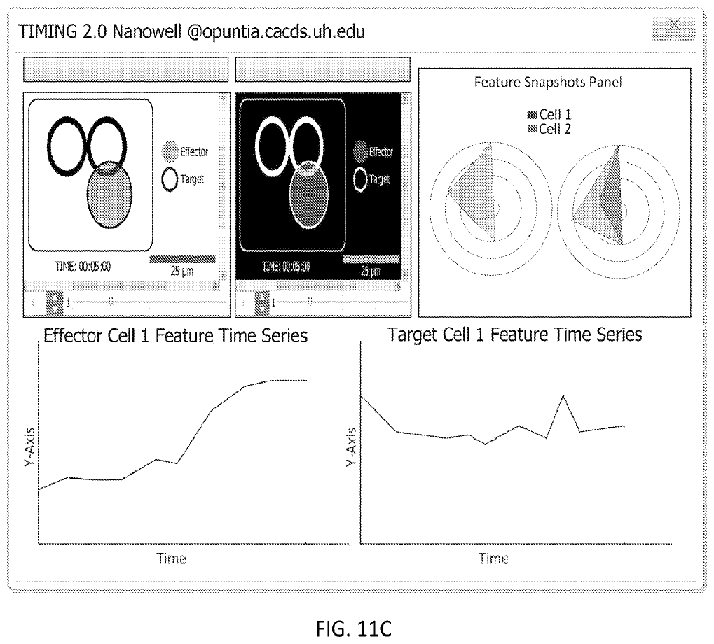

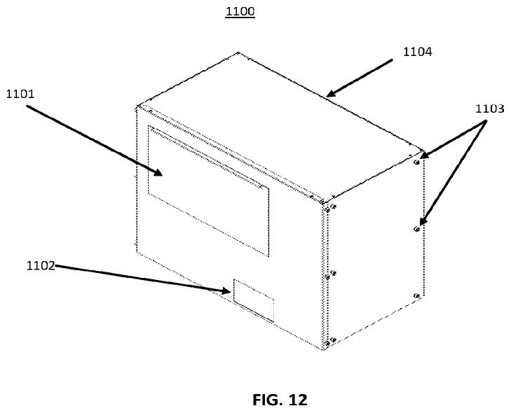

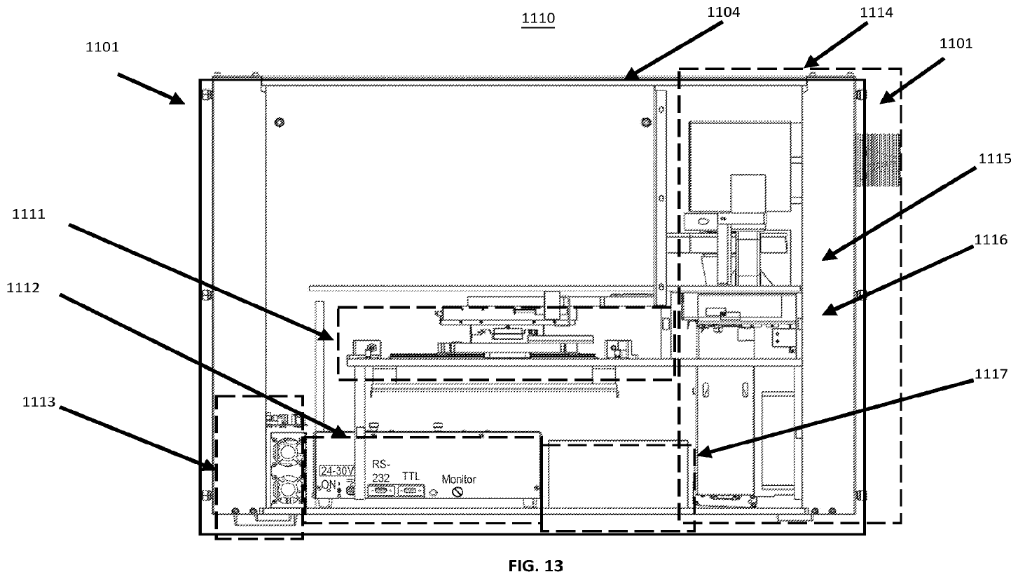

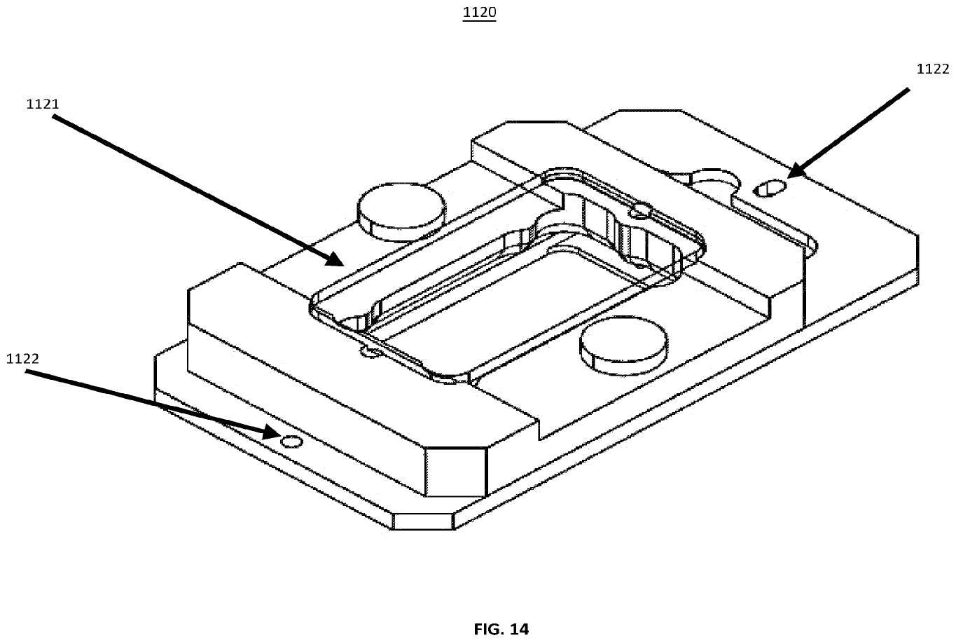

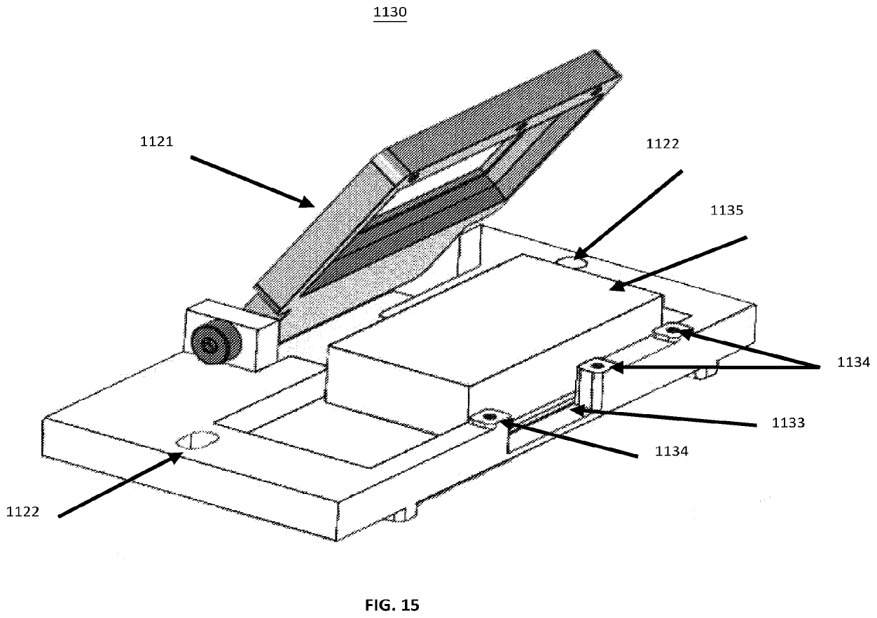

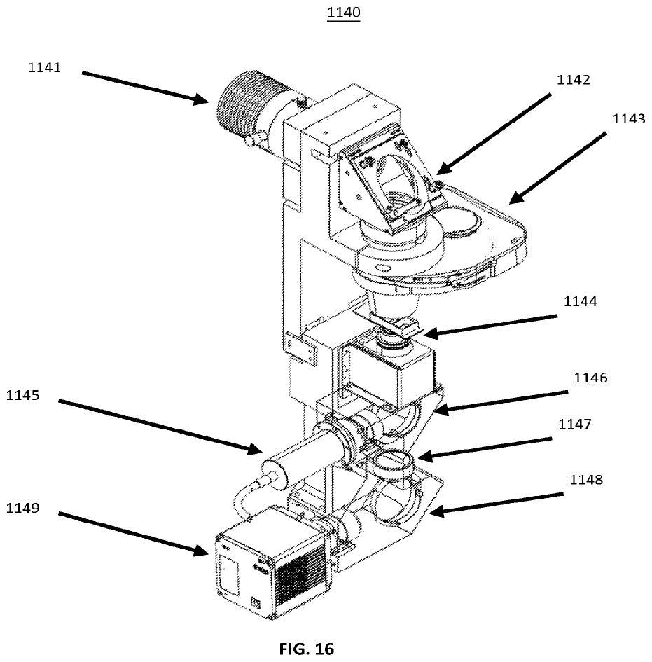

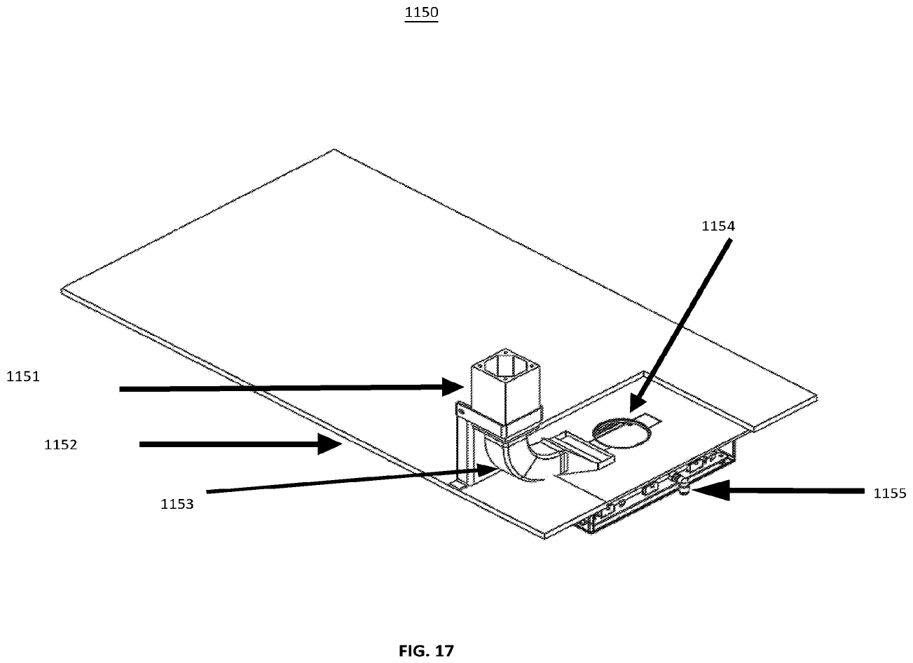

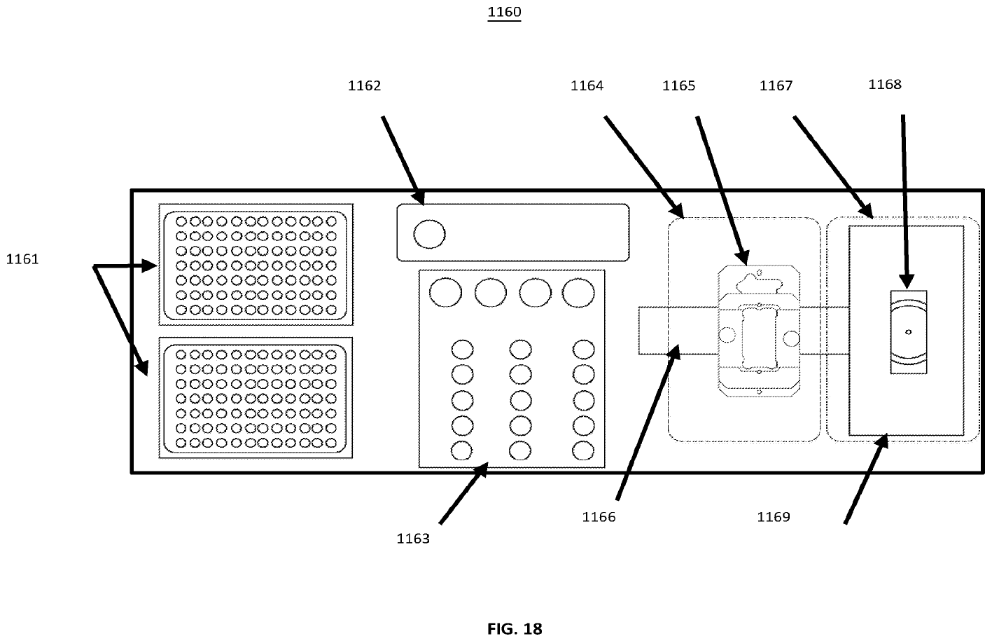

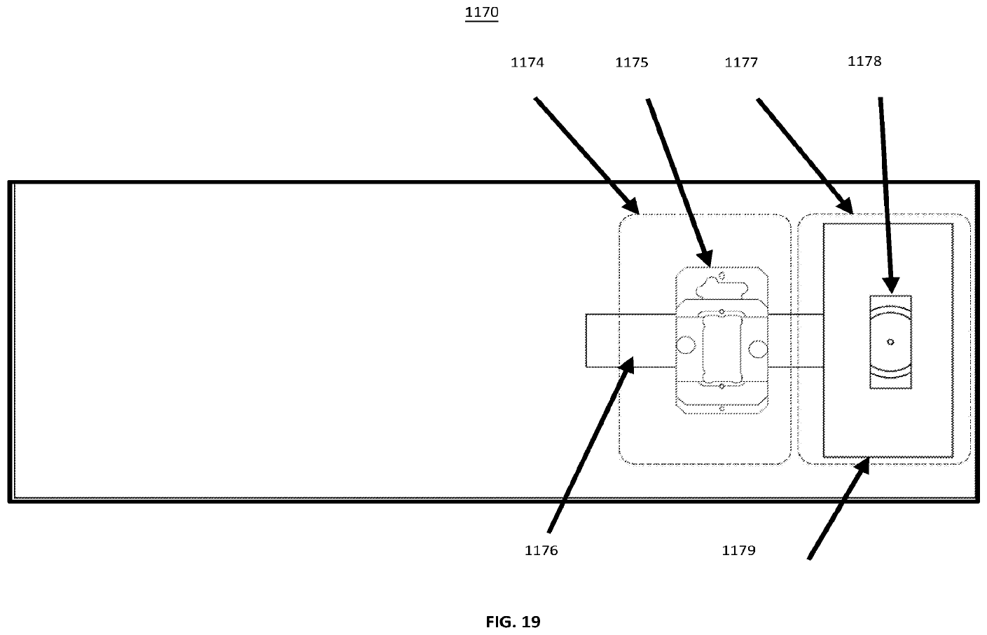

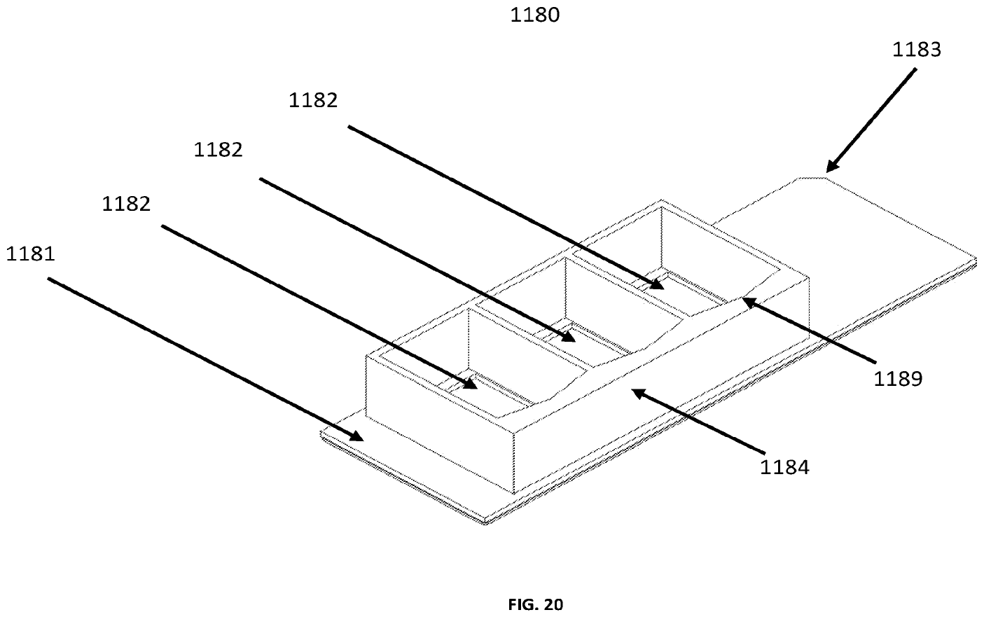

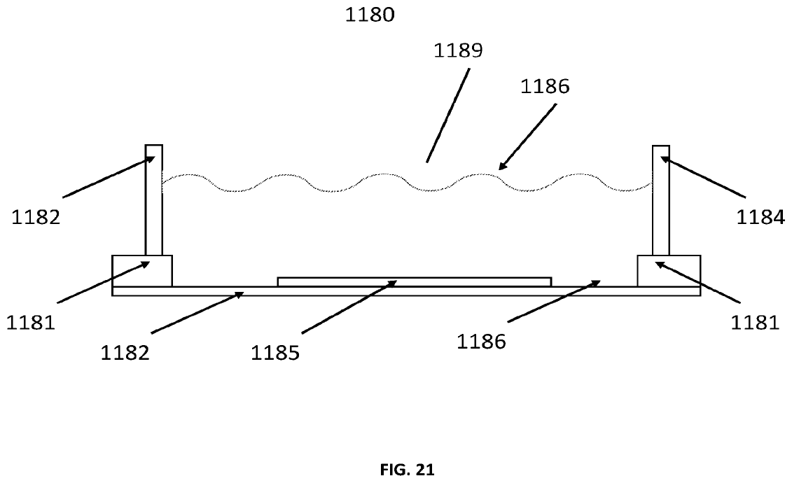

FIG. 11C illustrates a nanowell editor to allow for correction of the automated segmentation tracking errors in accordance with aspects of embodiments of the present invention.

FIG. 12 illustrates a system 1100 according to one or more embodiments of the present disclosure.

FIG. 13 illustrates a system 1110 in a cutaway view from the front according to one or more embodiments of the present disclosure.

FIG. 14 illustrates a system/device 1120 including an array carrier 1120 according to one or more embodiments of the present disclosure.

FIG. 15 illustrates a system/device 1130 including an array carrier 1120 according to one or more embodiments of the present disclosure.

FIG. 16 illustrates a system/device 1140 including a microscope assembly 1130 according to one or more embodiments of the present disclosure.

FIG. 17 illustrates a portion 1150 of a system that provides condensation prevention according to one or more embodiments of the present disclosure

FIG. 18 illustrates a system 1160 in cutaway view from the top, in accordance with one or more embodiments of the present disclosure.

FIG. 19 illustrates a system 1170 in cutaway view from the top according to one or more embodiments of the present disclosure.

FIG. 20 illustrates a device 1180 including one or more macrowells each containing an array of wells with dimensions in the micron range according to one or more embodiments of the present disclosure.

FIG. 21 illustrates a device 1180 in cutaway view from the side according to one or more embodiments of the present disclosure.

FIG. 22 illustrates a single block of wells 1190 from an array of wells 1185 according to one or more embodiments of the present disclosure (with neighboring blocks partially shown).

### DETAILED DESCRIPTION

Various detailed embodiments of the present disclosure, taken in conjunction with the accompanying figures, are disclosed herein; however, it is to be understood that the disclosed embodiments are merely illustrative. In addition, each of the examples given in connection with the various embodiments of the present disclosure is intended to be illustrative, and not restrictive.

Throughout the specification, the following terms take the meanings explicitly associated herein, unless the context clearly dictates otherwise. The phrases “in one embodiment” and “in some embodiments” as used herein do not necessarily refer to the same embodiment(s), though it may. Furthermore, the phrases “in another embodiment” and “in some other embodiments” as used herein do not necessarily refer to a different embodiment, although it may. Thus, as described below, various embodiments may be readily combined, without departing from the scope or spirit of the present disclosure.

In addition, the term “based on” is not exclusive and allows for being based on additional factors not described, unless the context clearly dictates otherwise. In addition, throughout the specification, the meaning of “a,” “an,” and “the” include plural references. The meaning of “in” includes “in” and “on.”

As used herein, the terms “and” and “or” may be used interchangeably to refer to a set of items in both the conjunctive and disjunctive in order to encompass the full description of combinations and alternatives of the items. By way of example, a set of items may be listed with the disjunctive “or”, or with the conjunction “and.” In either case, the set is to be interpreted as meaning each of the items singularly as alternatives, as well as any combination of the listed items.

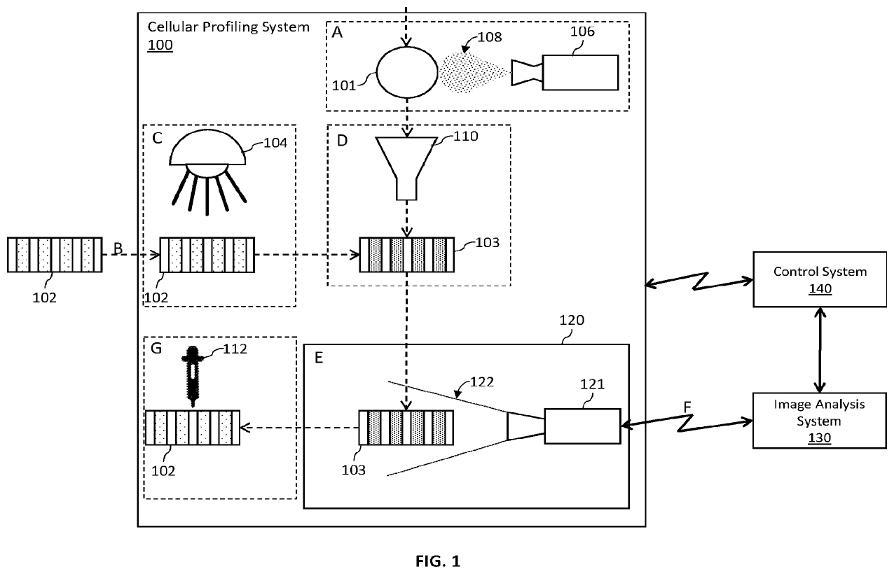

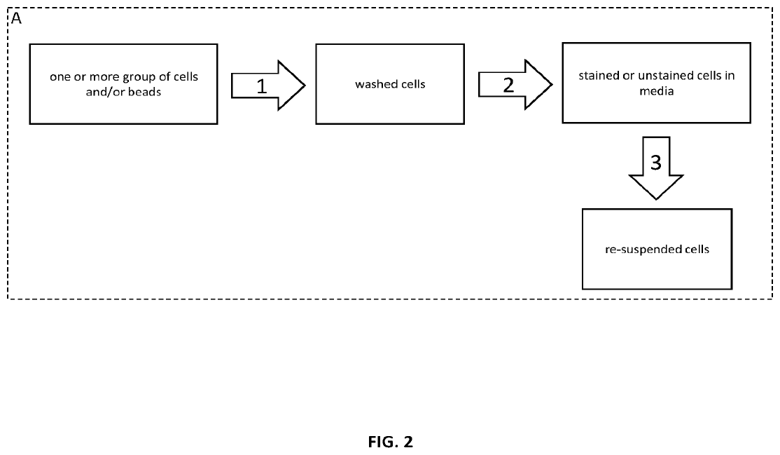

FIGS. 1 through 22 illustrate systems and methods of evaluating cellular activity of samples in an array of wells (e.g., micro- and/or nano-wells) using an imaging component and imaging analysis system. The following embodiments provide technical solutions and technical improvements that overcome technical problems, drawbacks and/or deficiencies in the technical fields involving omics and single-cell omics technologies. As explained in more detail, below, technical solutions and technical improvements herein include aspects of improved therapeutic potential through the identification of signatures of desired profiles lymphocytes (T cells, B cells and NK cells), neutrophils, and monocytes/macrophages cells, such as CAR+ T cells that can engage in serial (repeated) killing using improved cell preparation, imaging and image processing. For example, the improved cell preparation, imaging and image processing may enable evaluating different CAR designs for CAR T and other cell therapies to determine which candidates to move forward in development, predicting response based on the functional performance (and possibly integrating other molecular profiling) of allogeneic immune cell products, antibodies, vaccine-exposed cells, target cell lines and/or patient-derived immune cells, disease cells and other cells, including before manufacturing, after manufacturing or after treatment, evaluating the performance of manufactured products such as “release testing”, among features and functionality or any combination thereof.

Based on such technical features, further technical benefits become available to users and operators of these systems and methods. Moreover, various practical applications of the disclosed technology are also described, which provide further practical benefits to users and operators that are also new and useful improvements in the art.

FIG. 1 is a block diagram of a system for functional and molecular cell profiling in accordance with one or more embodiments of the present disclosure.

In some embodiments, a cellular profiling system 100 for functional and molecular cell profiling may be configured with multiple stages for sample retrieval and preparation, well array retrieval and preparation, well array loading, sample incubation and sample imaging, and sample retrieval from the well array. In some embodiments, to coordinate each stage with the sample and well array, a control system 140 may be provided in communication with the cellular profile system 100 to control devices of the cellular profile system 100 to effectuate each stage.

In some embodiments, the control system 140 may include a local processing system integrated with or in direct communication with the cellular profile system 100. In some embodiments, the processing system may include one or more compute resources for performing training for machine learning models (e.g., computer vision, image recognition, segmentation, etc.), and one or more additional compute resources for performing inferencing with the machine learning models. In some embodiments, the compute resources may include one or more central processing units (CPUs), one or more graphical processing units (GPUs), one or more neural processing units (NPUs), one or more resistive processing units (RPUs), among other compute resources or any combination thereof.

In some embodiments, a processing system may include, e.g., one or more processors, random access memory (RAM), read only memory (ROM), storage, among other computer hardware. In some embodiments, the control system 140 may include or be incorporated, partially or entirely into at least one personal computer (PC), laptop computer, ultra-laptop computer, tablet, touch pad, portable computer, handheld computer, palmtop computer, personal digital assistant (PDA), cellular telephone, combination cellular telephone/PDA, television, smart device (e.g., smart phone, smart table, etc.), mobile internet device (MID), messaging device, data communication device, and so forth.

In some embodiments, the control system 140 may include, e.g., a cloud or internet based service or other remote computing device. Accordingly, the control system 140 may control the devices of the cellular profile system 100 via a network connection to one or more remote servers and/or computers.

In some embodiments, the remote control system 140 may communicate with the cellular profile system 100 using a communication and/or networking protocol, such as, e.g., one or more suitable data communication networks (e.g., the Internet, satellite, etc.) and utilizing one or more suitable data communication protocols/modes such as, without limitation, IPX/SPX, X.25, AX.25, AppleTalk™, TCP/IP (e.g., HTTP), Bluetooth™, near-field wireless communication (NFC), RFID, Narrow Band Internet of Things (NBIOT), 3G, 4G, 5G, GSM, GPRS, WiFi, WiMax, CDMA, satellite, ZigBee, and other suitable communication modes. Various embodiments herein may include interactive posters that involve wireless, e.g., Bluetooth™ and/or NFC, communication aspects, as set forth in more detail further below.

In some embodiments, a “server” may refer to a service point which provides processing, database, and communication facilities. Some embodiments include a compute resource for algorithm training and a second for inference. By way of example, and not limitation, the term “server” can refer to a single, physical processor with associated communications and data storage and database facilities, or it can refer to a networked or clustered complex of processors and associated network and storage devices, as well as operating software and one or more database systems and application software that support the services provided by the server. Cloud servers are examples.

In some embodiments, the terms “cloud,” “Internet cloud,” “cloud computing,” “cloud architecture,” and similar terms may refer to at least one of the following: (1) a large number of computers connected through a real-time communication network (e.g., Internet); (2) providing the ability to run a program or application on many connected computers (e.g., physical machines, virtual machines (VMs)) at the same time; (3) network-based services, which appear to be provided by real server hardware, and are in fact served up by virtual hardware (e.g., virtual servers), simulated by software running on one or more real machines (e.g., allowing to be moved around and scaled up (or down) on the fly without affecting the end user). The aforementioned examples are, of course, illustrative and not restrictive.

In some embodiments, the control system 140 may include hardware components such as a processor, which may include local or remote processing components. In some embodiments, the processor may include any type of data processing capacity, such as a hardware logic circuit, for example an application specific integrated circuit (ASIC) and a programmable logic, or such as a computing device, for example, a microcomputer or microcontroller that include a programmable microprocessor. In some embodiments, the processor may include data-processing capacity provided by the microprocessor. In some embodiments, the microprocessor may include memory, processing, interface resources, controllers, and counters. In some embodiments, the microprocessor may also include one or more programs stored in memory.

Similarly, the control system 140 may include storage, such as local hard-drive, solid-state drive, flash drive, database or other local storage, or remote storage such as a server, mainframe, database or cloud provided storage solution.

In some embodiments, the control system 140 may implement computer engines for control of each stage of the cellular profiling system 100, such as, e.g., a sample preparation stage A, a well array retrieval stage B, a well array preparation stage C, a well array loading stage D, an incubation and imaging stage E, an image analysis stage F and a sample retrieval stage G. In some embodiments, the terms “computer engine” and “engine” identify at least one software component and/or a combination of at least one software component and at least one hardware component which are designed/programmed/configured to manage/control other software and/or hardware components (such as the libraries, software development kits (SDKs), objects, etc.).

Examples of hardware elements may include processors, microprocessors, circuits, circuit elements (e.g., transistors, resistors, capacitors, inductors, and so forth), integrated circuits, application specific integrated circuits (ASIC), programmable logic devices (PLD), digital signal processors (DSP), field programmable gate array (FPGA), logic gates, registers, semiconductor device, chips, microchips, chip sets, and so forth. In some embodiments, the one or more processors may be implemented as a Complex Instruction Set Computer (CISC) or Reduced Instruction Set Computer (RISC) processors; x86 instruction set compatible processors, multi-core, or any other microprocessor or central processing unit (CPU). In various implementations, the one or more processors may be dual-core processor(s), dual-core mobile processor(s), and so forth.

Examples of software may include software components, programs, applications, computer programs, application programs, system programs, machine programs, operating system software, middleware, firmware, software modules, routines, subroutines, functions, methods, procedures, software interfaces, application program interfaces (API), instruction sets, computing code, computer code, code segments, computer code segments, words, values, symbols, or any combination thereof. Determining whether an embodiment is implemented using hardware elements and/or software elements may vary in accordance with any number of factors, such as desired computational rate, power levels, heat tolerances, processing cycle budget, input data rates, output data rates, memory resources, data bus speeds and other design or performance constraints.

In some embodiments, to each of the sample preparation stage A, a well array retrieval stage B, the well array preparation stage C, the well array loading stage D, the incubation and imaging stage E, the image analysis stage F and the sample retrieval stage G may include computer engines having distinct hardware and software components, shared hardware components, shared software components, or any combination of shared and distinct hardware components and software components across the stages.

In some embodiments, at the sample preparation stage A, a sample 101 of cells may be received and prepared. In some embodiments, the sample 101 may include one cell or a collection of cells. For a collection of cells, the sample 101 may include one or more groups of cells. In some embodiments, the sample 101 may additionally or alternatively include beads (e.g., for cytokine capture) or coated antibodies.

In some embodiments, to prepare the sample 101, an applicator 106 may be controlled by the control system 140 to apply one or more reagent 108 onto the sample to mark cells and keep the cells alive. For example, in some embodiments, the sample 101 may include adherent cells, cells in suspension, or in any other suitable sample form. In some embodiments, for adherent cells, the sample preparation stage A may include a cell detacher, such as, e.g., Corning Cellstripper or other similar cell detachment solution and/or device. In some embodiments, the detacher may be applied with the applicator 106 or with a separation application device or a combination thereof. In some embodiments, the applicator 106 regulates temperature, number of washes, and incubation time based on the requirements of reagent 108.

In some embodiments, the applicator 106 may include any suitable application device or devices for applying one or more preparation substances 108 to the sample 101 to prepare the sample for analysis. For example, the preparation substance 108 may include, e.g., a reagent such as a membrane dye to stain the outer membrane of cells (e.g., PKH26 red, PKH67 green), a nucleus stain for staining the nucleus of cells (e.g., Hoechst), a cleaning substance, or any other suitable preparation substance or any combination thereof.

In some embodiments, upon completing preparation of the sample 101 using the preparation substance 108 applied by the application 106, the sample 101 may be moved to a sample loader 110. In some embodiments, the control system 140 may control a transport mechanism to transport the sample from the sample preparation stage A to the sample loader 110 of the well array loading stage D. In some embodiments, the transport mechanism may include, e.g., a robotic arm with a pincer or other grasping device, a conveyer belt, a movable platform, or any other suitable transport mechanism.

In some embodiments, concurrently with the sample preparation, or before or after cell preparation, a well array 102 may be provided to the cellular profiling system 100. In some embodiments, the well array retrieval stage B may retrieve the provided well array 102. In some embodiments, the well array retrieval stage B may include a suitable deposit area and/or transport mechanism. For example, any suitable device or set of devices may be employed for to receive a cassette, slide, plate, petri dish or other element 1180 containing one or more array or other element that will contain cells (e.g., which includes one or more array of nano- or micro-wells), such as a stage which would be presented to the user (e.g., a drawer slides/can be slide out, or a door opens/is opened, and user can place the device (plate, slide, etc) on the stage. In some embodiments, closing the drawer or door may signal the control system 140 that the well array 102 has been received. In some embodiments, the rather than presentation by a user, the well array 102 may be automatically retrieved from an inventor by, e.g., a robotic arm or robotic picker, a feeder, a conveyer, a moveable platform, or other automated component or any combination thereof. In some embodiments, the automated component(s) may be controlled by the control system 140 to identify and retrieve the well array 102.

In some embodiments, the well array 102 may be prepared at the well array preparation stage C. In some embodiments, the well array preparation stage C may include one or more suitable devices and/or components for cleaning and/or coating the well array 102 in preparation to receive the sample 101 without contaminating the sample 101. In some embodiments, the well array preparation stage C may include, e.g., a plasma oxidizer 104 to oxidize the well array 102, create a hydrophilic surface on the well array, facilitate the sample loading by sample loader 110 and/or sterilize the well array 102 from contaminating cells and substances. In some embodiments, the plasma oxidizer 104 may be controlled by the control system 140 to perform plasma oxidation of the well array 102, e.g., within a vacuum chamber. For example, the control system 140 may control the vacuum chamber to activate a pump for, e.g., one minute to vacuum the chamber at −10 to −20 PSI.

In some embodiments, the vacuum chamber may include a vacuum pump (e.g., a Harrick Plasma PDC-VPE or similar) for controlling the pressure in the vacuum chamber. In some embodiments, the vacuum pump may depressurize the vacuum chamber upon entry of the sample 101, e.g., to a pressure −10 pounds per square inch (psi) or below, −20 psi or below, between −10 and −20 psi, or other suitable vacuum pressure for cleaning the well array 102 to prevent contamination of the sample 101.

In some embodiments, once the vacuum chamber has completed the vacuum, the plasma oxidizer 104 may be turned on for any suitable period of time to ensure that the array is clean, e.g., about 1 minute or more. In some embodiments, the vacuum chamber may include a plasma oxidizer 104 with a suitable a plasma cleaner (e.g., a Harrick Plasma PDC-32G or similar) for effectuating plasma cleaning the well array 102 to prevent contamination of the sample 101.

In some embodiments, upon cleaning the well array 102, the cellular profiling system 100 may transport the well array 102 to the well array loading stage D. In some embodiments, the control system 140 may control a transport mechanism to transport the well array 102 from the array preparation stage C to the well array loading stage D. In some embodiments, the transport mechanism may include, e.g., a robotic arm with a pincer or other grasping device, a conveyer belt, a movable platform, or any other suitable transport mechanism.

In some embodiments, upon the sample 101 and the well array 102 entering the well array loading stage D, the control system 140 may instruct the sample loader 110 to deposit the sample 101 into one or more wells of the well array 102, and produce a filled well array 103.

In some embodiments, the sample loader 110 may include any suitable device or component to provide the sample 101 to the well array 102, and to optimally capture the sample 101 into each well. In some embodiments, the sample loader 110 may include, e.g., an automated pipetting system, microfluidic devices, flow cytometry devices, etc. In some embodiments, the sample loader 110 may use a robotic device (a liquid handler with arms for moving and tilting the cassette) to automatically perform the pipetting and loading cells of the sample 101 in an optimized manner to distribute cells and media or reagents across the array of wells (e.g., nanowells or microwells) of the well array 102.

In some embodiments, the sample loader 110 may allow for multiple effector to target (E:T ratios). A device for increasing the accuracy of cell loading can improve the distributed evaluation. Improvement of a high throughput evaluation can be done by optimized method for cell loading, faster image acquisition, image analysis and processing, improvement of cell labeling, improvement in label-free cell detection, etc.

In some embodiments, upon filling the well array 102, the filled well array 103 may be transported to the incubation and imaging stage E. In some embodiments, the control system 140 may control a transport mechanism to transport the filled well array 103 from the well array loading stage D to the incubation and imaging stage E. In some embodiments, the transport mechanism may include, e.g., a robotic arm with a pincer or other grasping device, a conveyer belt, a movable platform, or any other suitable transport mechanism.

Incubator 120 may be controlled by the control system 140 to incubating sample in the filled well array 103, e.g., at a constant temperature (where the temperature is cell dependent but usually 37 degrees, and were incubation can be as short as several minutes to 24 hours or longer, depending on the experiment).

In some embodiments, while incubating, the filled well array 103 may be maintained in a position within a field of view 122 of an imaging device 121. In some embodiments, the imaging device 122 may include any suitable device, component or system for capturing images through time of the sample in the filled well array 103 to monitor cell behaviors and interactions. In some embodiments, the imaging device 121 may include multiple cameras and/or other imaging elements such that more than one area of the array can be imaged in parallel and/or one or more arrays or sub-arrays can be imaged in parallel, by capturing individual images at the same time and/or in sequence so as to improve the throughput. For example, three cameras may capture images from each of three arrays contained on or in a cassette, slide, plate, petri dish or other element such that the user does not need to run three experiments sequentially or on three different devices.

In some embodiments, the imaging device 121 may capture images of the cells at time points (e.g., a sequence of still images) or continuously (e.g., a continuous sequence of still images or a video feed). In some embodiments, the imaging device 121 may capture brightfield, darkfield and/or phase contrast images. In some embodiments, while capturing images, the filled well array 103 may be held in the position in the field of view 122 for incubation in an atmosphere that fosters incubation while facilitating precise imaging of the cells of the sample. For example, in some embodiments, the incubation chamber 120 may maintain an environment with sufficient CO2 (where a sensor is used to measure and set the CO2 level consistent throughout the experiment at a CO2 level that is usually 5%), potentially including multi-color imaging and/or high-speed changing between color channels (e.g., at millisecond timescale) where preferred lasers with specific wavelengths in the visible light spectra are used such as with pre-selected filters (395/25, 440/20, 470/24, 510/25 550/15, 575/25 and 640/30).

In some embodiments, the imaging device 121 may output image files representing, e.g., a sequence of still photographs and/or frames of a continuous video feed. In some embodiments, the image files may be in a lossy or lossless raster format such as, e.g., Joint Photographic Experts Group (JPEG), Tagged Image File Format (TIFF), Portable Network Graphics (PNG), Exchangeable image file format (Exif), Graphics Interchange Format (GIF), Windows bitmap (BMP), portable pixmap (PPM) or other formats from the Netpbm format family, WebP, High Efficiency Image File Format (HEIF), BAT, Better Portable Graphics (BPG), or a lossy or lossless vector format such as, Computer Graphics Metafile (CGM), Gerber (RS-274X), Scalable Vector Graphics (SVG), or other formats and combinations thereof. The file format of the image files may depend on the imaging device 121, such as the format used by a digital camera or smartphone, which can vary from device to device.

In some embodiments, the image files may be provided to an image analysis system 130 of an image analysis stage F. In some embodiments, the image analysis system 130 may include a local processing system integrated with or in direct communication with the cellular profile system 100. In some embodiments, a processing system may include, e.g., one or more processors, random access memory (RAM), read only memory (ROM), storage, among other computer hardware. In some embodiments, the image analysis system 130 may include or be incorporated, partially or entirely into at least one personal computer (PC), laptop computer, ultra-laptop computer, tablet, touch pad, portable computer, handheld computer, palmtop computer, personal digital assistant (PDA), cellular telephone, combination cellular telephone/PDA, television, smart device (e.g., smart phone, smart table, etc.), mobile internet device (MID), messaging device, data communication device, and so forth.

In some embodiments, the image analysis system 130 may include, e.g., a cloud or internet based service or other remote computing device. Accordingly, the image analysis system 130 may control the devices of the cellular profile system 100 via a network connection to one or more remote servers and/or computers.

In some embodiments, the remote image analysis system 130 may communicate with the cellular profile system 100 using a communication and/or networking protocol, such as, e.g., one or more suitable data communication networks (e.g., the Internet, satellite, etc.) and utilizing one or more suitable data communication protocols/modes such as, without limitation, IPX/SPX, X.25, AX.25, AppleTalk™, TCP/IP (e.g., HTTP), Bluetooth™, near-field wireless communication (NFC), RFID, Narrow Band Internet of Things (NBIOT), 3G, 4G, 5G, GSM, GPRS, WiFi, WiMax, CDMA, satellite, ZigBee, and other suitable communication modes. Various embodiments herein may include interactive posters that involve wireless, e.g., Bluetooth™ and/or NFC, communication aspects, as set forth in more detail further below.

In some embodiments, the image analysis stage F may include an image analysis system 130 to extract data, perform analysis, receive parameters, output data/analysis/visualizations, and/or communicate with external devices including cloud-based software. In some embodiments, the image analysis system 130 is able to recognize one or more, cassette, slide, plate, petri dish or other element containing one or more array or other element that will contain cells by reading a barcode, reading a QR code, reading an RFID chip, imaging the design of the array of wells including features such as the rotation of specific wells that may identify the position of wells and/or blocks on the element containing one or more array, and/or otherwise identifying the product in order to validate authenticity of the product, identify the type of product and/or its dimensions or other features, recognize an analysis credit, track sample(s), and/or track experiments.

In some embodiments, image analysis system 130 may implement analysis algorithms that are comprised of nanowell detection (local maximum clustering algorithm), nanowell localization (e.g., normalized cross-correlation based template fitting), pre-processing (background subtraction and correction for spectral overlap between emission spectra of multiple fluorescence dye), nanowell prioritization (wherein a subset of nanowells and/or blocks of nanowells are analyzed to determine which nanowells and/or blocks of nanowells should be imaged and/or analyzed in later steps, for example based on preferred numbers of cells per nanowell, ratios of two or more different cell types, or other criteria), cell counting (normalized multi-threshold distance map (NMTDM) algorithm), cell tracking, and cell-cell contact algorithm.

In some embodiments, the imaging device 121 generates an array of multi-channel videos of up to 200 000 nanowells, and typically 5000 to 20 000 nanowells per sample, sampled up to 60 min apart, and typically 1 to 15 minutes apart. The algorithms of the image analysis system 130 may take advantage of nanowells that are rotated by 45° at known locations on the array to uniquely locate individual wells in an array.

In some embodiments, the image analysis system 130 may implement algorithms that include, e.g., normalized cross-correlation (NCC) for image registration, Fourier implementation to normalize in the spatial domain, local maximum clustering algorithm on the best-fitting NCC response to detect well centers, leveling to correct illumination variations by subtracting the local background estimated at each pixel using a Gaussian kernel, unmixing by a linear inverse method, image smoothing using a median filter (with radius rm˜=3) while preserving cell boundaries, cell body detection with a normalized multi-threshold distance map (NMTDM) that averages distance maps corresponding to multiple thresholds, local maxima clustering for cell detection, normalized spectral clustering of image pixels to re-segment cells, a directed graph for confinement-constrained cell tracking, soft cell interaction measure CI for quantifying the interaction of a cell with its surrounding cells, or a regional convolutional neural network for cellular and subcellular detection and segmentation.

In some embodiments, the image analysis system 130 parameters are used to identify and filter out specific observations. In some embodiments, the parameters can be set at one or more predetermined standard values, or can be adjusted by the user. The observation can be for example cell death (identified by death marker threshold), or cell morphology (identified by cell size, shape, etc).

In some embodiments, the image analysis system 130 generate outputs are the image sequences of cell-cell interaction and summarized table for multiple parameters measured by TIMING. These tables may include multiple parameters used for comparison between samples. The comparisons are shown in form of bar plots, scatter plots, survival curves, etc.

In some embodiments, upon completion of incubation and imaging, the filled well array 103 may be transported to the sample retrieval stage G. In some embodiments, the control system 140 may control a transport mechanism to transport the well array 102 from the incubation and imaging stage E to the sample retrieval stage G. In some embodiments, the transport mechanism may include, e.g., a robotic arm with a pincer or other grasping device, a conveyer belt, a movable platform, or any other suitable transport mechanism.

In some embodiments, the sample retrieval stage G may include, e.g., a sample picker 112 controlled by the control system 140 to retrieve one or more cells from one or more wells of the filled well array 103. In some embodiments, the sample picker 112 may include, e.g., a glass capillary with the diameter size of the well of the filled well array 103. In some embodiments, the control system 140 may be preprogrammed with the location of each well on the array and the cells such that the control system 140 may selectively instruct the sample picker 112 to retrieve particular cells from the preprogrammed locations, e.g. based on the output from the image analysis system 130, based on user selection, or by any other suitable selection methodology or any combination thereof.

FIG. 2 illustrates a flowchart of an illustrative methodology cell preparation for functional and molecular cell profiling in accordance with one or more embodiments of the present disclosure.

In some embodiments, the sample preparation stage A may include cell staining, cell washing, media application, and cell suspension.

In some embodiments, washing a number of cells may include application, e.g., via the applicator 106, may include applying a solution for cleaning the sample of cells and/or beads. For example, one million each effector and target cells may be provided in a solution such as phosphate-buffered saline (PBS) and resuspending in marker reagents.

In some embodiments, target and effector cells may be stained with two markers. Example staining reagents are PKH67 Green for effector and PKH26 Red for target. Alternatively, algorithms may use label-free identification and tracking of cells.

In some embodiments, cells and/or beads may be resuspended in media (e.g., from the applicator 106) which that may be compatible with AnxV staining (death marker). An example media is IMDM 10% FBS (I10). The final concentration of cells may be around 1.2 Million cell/ml.

In some embodiments, the sample preparation stage A may include a centrifugation system (or alternative) for washing the cells during the staining steps. This step optimally includes a cell counter built in the instrument to provide a desired concentration. Procedures for staining cells may employ manipulating and pipetting small amounts of reagents up to 10 ul. A liquid handler may be employed. Procedures for staining cells may employ specific time and temperature. An incubator being able to control the temperature and time may be employed. Procedures for staining cells may employ mixing the reagents with cells. A mixer or vortex may be employed.

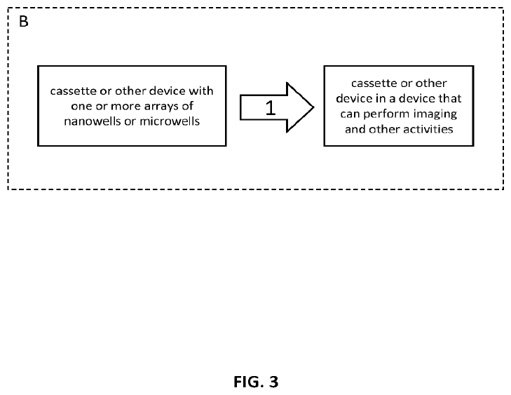

FIG. 3 illustrates a flowchart of an illustrative methodology cassette loading for functional and molecular cell profiling in accordance with one or more embodiments of the present disclosure.

In some embodiments, the well array retrieval stage B may include receiving a well array including, e.g., a cassette, slide, plate, petri dish or other element containing one or more array or other element that will contain cells (e.g., which includes one or more array of nano- or micro-wells). In some embodiments, receiving the well array may include, e.g., loading (manually or automatically) the well array onto a stage of the cellular profiling system 100. In some embodiments, a robotic arm, for example, may pick the well array from a stock of well arrays based on automated control, e.g., from the control system 140. In some embodiments, closing a door to the stage or actuating the stage to move the well array to within the cellular profile system 100 may trigger the well array preparation stage C.

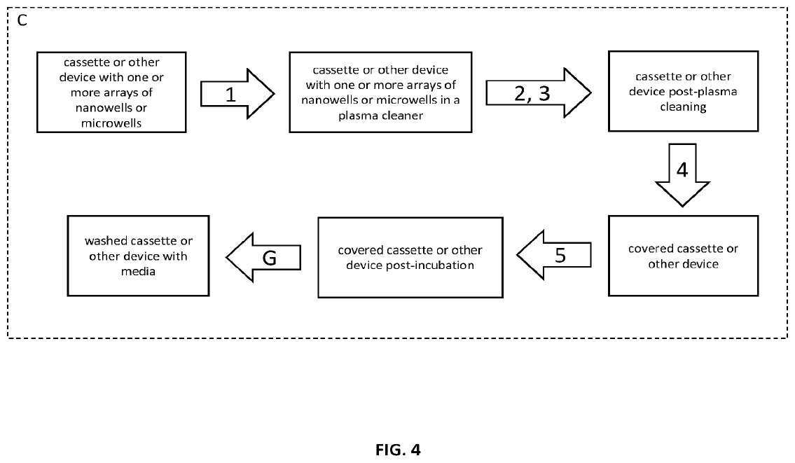

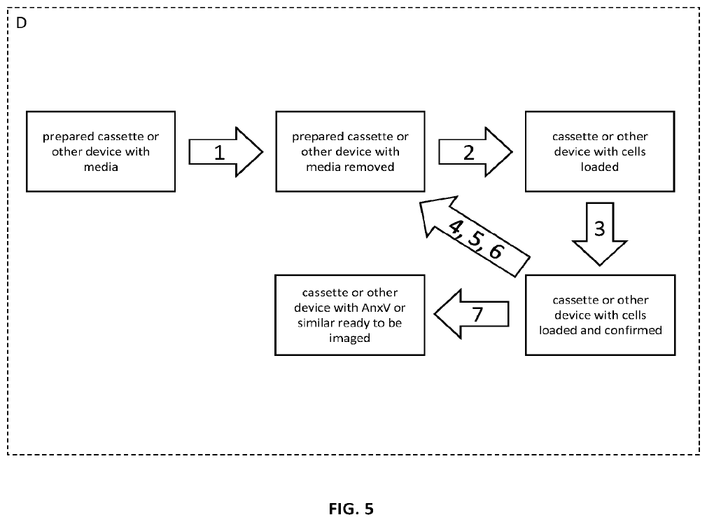

FIG. 4 illustrates a flowchart of an illustrative methodology for cassette preparation for functional and molecular cell profiling in accordance with one or more embodiments of the present disclosure.

In some embodiments, the well array preparation stage C may include placing a cassette, slide, plate, petri dish or other element containing one or more array or other element that will contain cells in a plasma cleaner. In some embodiments, the array may include, e.g., any suitable well or well array for holding a sample, such as, e.g., a 1 cm×1 cm chip in each well, or any other suitable dimension of chip.

In some embodiments, the plasma cleaner may be placed in a vacuum chamber. In some embodiments, the vacuum chamber may, for example by turning on a pump for one minute to vacuum the chamber. In some embodiments, once the vacuum chamber has completed the vacuum, the plasma cleaner may be turned on for any suitable period of time to ensure that the array is clean, e.g., about 1 minute or more.

In some embodiments, to preserve the sterility after plasma cleaning, the array may be covered in a suitable material to seal the array. For example, the array may be covered with media (such as R10) or poly(L-lysine)-g-poly(ethylene glycol) (PLL-g-PEG) solution. In some embodiments, an additional incubation step may be implemented, such as where PLL-g-PEG is used to cure the PLL-g-PEG. For example, incubation when using PLL-g-PEG may include, e.g., incubating the array at 37 degrees for 20 minutes.

In some embodiments, the array may then be washed with media and covered with media (such as R10). In some embodiments, it is optimal to rapidly transfer the chip from plasma cleaning to washing, and to cover the cassette or other element quickly with media.

FIG. 5 illustrates a flowchart of an illustrative methodology cell/bead loading into a cassette for functional and molecular cell profiling in accordance with one or more embodiments of the present disclosure.

In some embodiments, the well array loading stage D may include loading a sample of cells into the prepared well array (e.g., prepared as described above with reference to FIG. 1 and/or FIG. 4). In some embodiments, the sample may include cells and/or beads.

In some embodiments, well array loading may include removing media from the well array to enable filling with the sample.

In some embodiments, cells and/or beads of the sample may be deposited with the wells of the well array upon removal of the media. For example, the deposition may include depositing 40 microliters of effector cells on the chip, waiting a period of time (for example two to three minutes), and confirming loading optically such as with a microscope.

In some embodiments, upon loading, the well array filled with the sample may be washed, for example washing from top to bottom with media (e.g., R10) and confirming optically or otherwise such as with a microscope or through other means.

In some embodiments, the deposition and cleaning steps may be repeated until all cells and/or beads and/or groups thereof are deposited into the well array.

In some embodiments, once the well array is ready for imaging, the well array may be washed and/or applied with a media or reagent, for example wash with R10 and add AnxV in 110.

The steps above may include other elements that are intended to be evaluated with or without cells and or beads, such as emulsion droplets. Procedures for loading cells and/or beads (and/or other elements) may employ handling and pipetting small amounts of reagents, for example up to 20-100 ul, and so a liquid handler may be required. Procedures for loading cells and/or beads (and/or other elements) may employ several washing steps. A mechanical device for tilting the cassette, slide, plate, petri dish or other element containing one or more array or other element that contains cells may be required. Procedures for loading cells may require mixing reagents with cells and/or beads (or other elements). A mixer or vortex may be employed. Procedures for loading cells may employ depositing cells and/or beads (and/or other elements) on the right spot on the chip. The device needs to be equipped with a device that can spot the chip in the well of the 6-well plate and deposit correctly.

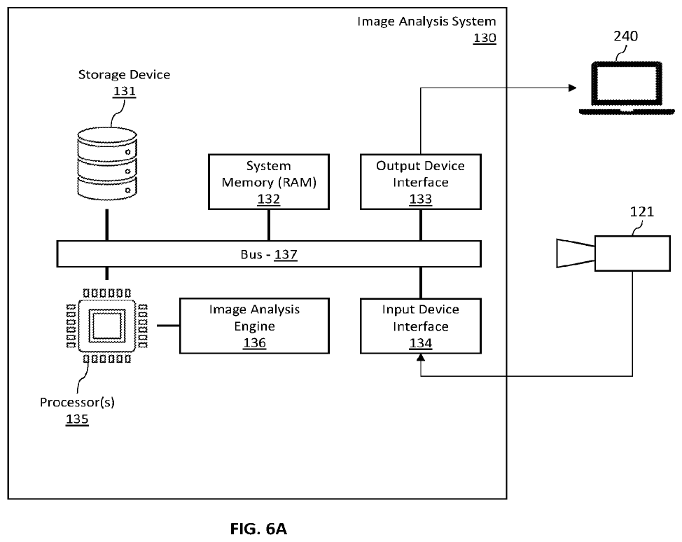

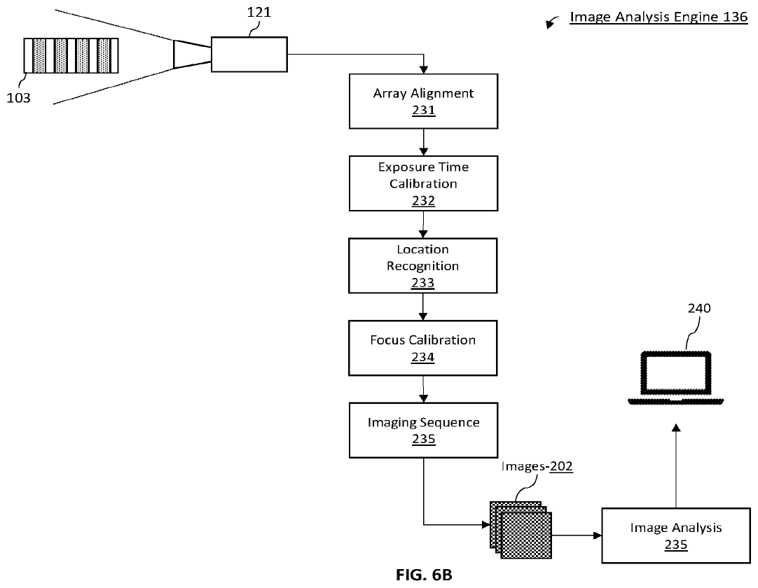

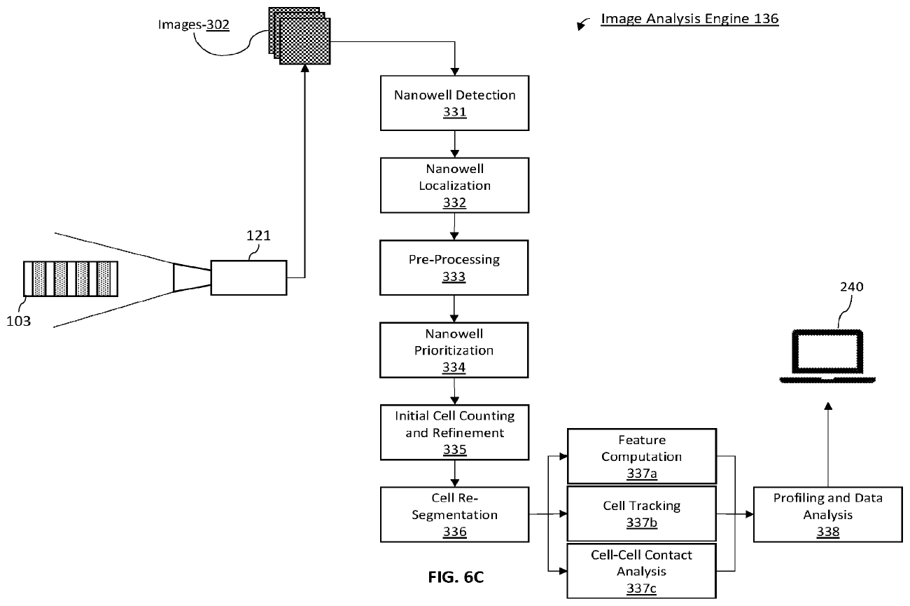

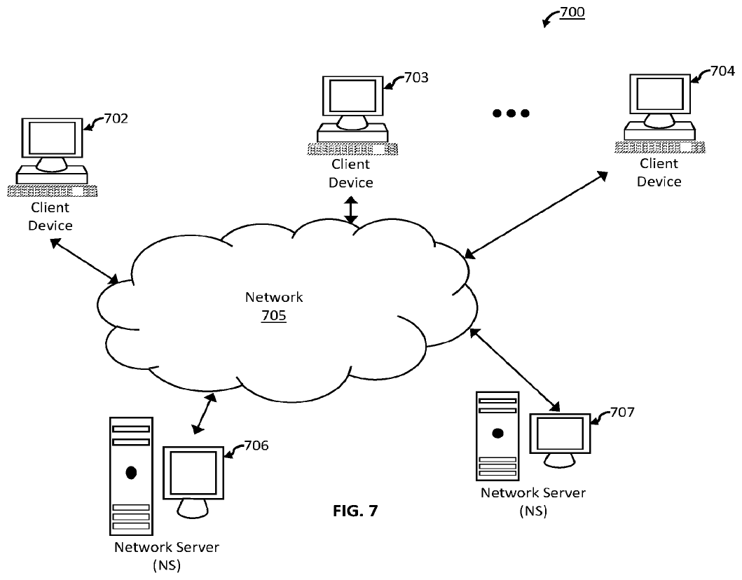

FIG. 6A is a block diagram of an image analysis system for functional and molecular cell profiling in accordance with one or more embodiments of the present disclosure.

In some embodiments, as described above, the imaging device 121 may communicate to the image analysis system 130 images of the samples 101 in the filled well array 103 during incubation of the sample 101. In some embodiments, the imaging device 121 may provide the images to the image analysis system 130 in a continuous stream or in one or more batches via an input device interface 134.

In some embodiments, the image analysis system 130 may receive the images and analyze the images with an image analysis engine 136 implemented by one or more processor(s) 135. In some embodiments, the image analysis engine 136 may process the images to, e.g., implement algorithms for nanowell detection (e.g., local maximum clustering algorithm), pre-processing (e.g., background subtraction and correction for spectral overlap between emission spectra of multiple fluorescence dye), cell counting (e.g., normalized multi-threshold distance map (NMTDM) algorithm), cell tracking, and cell-cell contact algorithm.

As a result, in some embodiments, the image analysis engine 136 may generate sample analyses that profile omics and multi-omics related to the sample 101. As a result, in some embodiments, the omics may be provided to a computing device 240 to display the results, e.g., via one or more visualizations (e.g., see, FIGS. 11A, 11B and 11C described below).

In some embodiments, the output device interface 133 and the input device interface 134 may each include any suitable hardware and/or networking interface. In some embodiments, hardware interfaces may include, e.g., a data connection port and/or protocol, including, e.g., Universal Serial Bus (USB), DisplayPort, Host-to-Host Communications, PCI Express, Thunderbolt, Firewire, VitualLink, High-Definition Multimedia Interface (HDMI), Mobile High Definition Link (MHL), Lightning, among others or any combination thereof. In some embodiments, networking interfaces may include, e.g., any suitable wired and/or wireless data communication hardware and/or protocol, including, e.g., Bluetooth, Wifi, Zigbee, local area network (LAN), wireless LAN, Zigbee, Z-Wave, cellular communications, among others or any combination thereof.

In some embodiments, the images may be access by the processor(s) 135 via a bus 137. In some embodiments, the bus 137 may include any suitable communication system that transfers data between components inside the image analysis system 130, include an internal data bus, memory bus, system bus, address bus, front-side bus, or other internal bus or any combination thereof. In some embodiments, examples of the bus 139 may include, e.g., PCI express, small computer system interface (SCSI), parallel AT attachment (PATA), serial AT attachment (SATA), HyperTransport™, InfiniBand™, Wishbone, Compute Express Link (CXL), among others or any combination thereof.

In some embodiments, to perform the image analyses, the processor(s) 135 may access the images and load the image analysis engine 136, e.g., from a system memory 132. In some embodiments, the system memory 132 may include any suitable random access memory, such as static RAM and/or dynamic RAM. In some embodiments, image analysis engine 136 may be retrieved from a storage device 131 via the bus 137, e.g., as an application, set of applications or other software application and/or software functions and loaded into the system memory 132. Accordingly, the processor(s) 135 may execute the software functions of the image analysis engine 136 to process each image of images.

In some embodiments, the data storage solution of the storage device 131 may include, e.g., a suitable memory or storage solutions for maintaining electronic data representing the activity histories for each account. For example, the data storage solution may include database technology such as, e.g., a centralized or distributed database, cloud storage platform, decentralized system, server or server system, among other storage systems. In some embodiments, the data storage solution may, additionally or alternatively, include one or more data storage devices such as, e.g., a hard drive, solid-state drive, flash drive, or other suitable storage device. In some embodiments, the storage device may, additionally or alternatively, include one or more temporary storage devices such as, e.g., a random-access memory, cache, buffer, or other suitable memory device, or any other data storage solution and combinations thereof.

In some embodiments, the image analysis engine 138 may include at least one software component and/or a combination of at least one software component and at least one hardware component which are designed/programmed/configured to manage/control other software and/or hardware components (such as the libraries, software development kits (SDKs), objects, etc.).

In some embodiments, software of the image analysis engine 138 may include any suitable combination of logical algorithms and/or machine learning algorithms for nanowell detection (e.g., local maximum clustering algorithm), pre-processing (e.g., background subtraction and correction for spectral overlap between emission spectra of multiple fluorescence dye), cell counting (e.g., normalized multi-threshold distance map (NMTDM) algorithm), cell tracking, and cell-cell contact algorithm.

In some embodiments, the image analysis engine 138 may be configured to utilize one or more exemplary AI/machine learning techniques chosen from, but not limited to, decision trees, boosting, support-vector machines, neural networks, nearest neighbor algorithms, Naive Bayes, bagging, random forests, and the like. In some embodiments and, optionally, in combination of any embodiment described above or below, an exemplary neutral network technique may be one of, without limitation, feedforward neural network, radial basis function network, recurrent neural network, convolutional network (e.g., U-net) or other suitable network. In some embodiments and, optionally, in combination of any embodiment described above or below, an exemplary implementation of Neural Network may be executed as follows:
a. define Neural Network architecture/model,
b. transfer the input data to the exemplary neural network model,
c. train the exemplary model incrementally,
d. determine the accuracy for a specific number of timesteps,
e. apply the exemplary trained model to process the newly-received input data,
f. optionally and in parallel, continue to train the exemplary trained model with a predetermined periodicity.

In some embodiments and, optionally, in combination of any embodiment described above or below, the exemplary trained neural network model may specify a neural network by at least a neural network topology, a series of activation functions, and connection weights. For example, the topology of a neural network may include a configuration of nodes of the neural network and connections between such nodes. In some embodiments and, optionally, in combination of any embodiment described above or below, the exemplary trained neural network model may also be specified to include other parameters, including but not limited to, bias values/functions and/or aggregation functions. For example, an activation function of a node may be a step function, sine function, continuous or piecewise linear function, sigmoid function, hyperbolic tangent function, or other type of mathematical function that represents a threshold at which the node is activated. In some embodiments and, optionally, in combination of any embodiment described above or below, the exemplary aggregation function may be a mathematical function that combines (e.g., sum, product, etc.) input signals to the node. In some embodiments and, optionally, in combination of any embodiment described above or below, an output of the exemplary aggregation function may be used as input to the exemplary activation function. In some embodiments and, optionally, in combination of any embodiment described above or below, the bias may be a constant value or function that may be used by the aggregation function and/or the activation function to make the node more or less likely to be activated.

In some embodiments, the computing device 140 may include or be incorporated, partially or entirely into at least one personal computer (PC), laptop computer, ultra-laptop computer, tablet, touch pad, portable computer, handheld computer, palmtop computer, personal digital assistant (PDA), cellular telephone, combination cellular telephone/PDA, television, smart device (e.g., smart phone, smart tablet or smart television), mobile internet device (MID), messaging device, data communication device, and so forth.

FIG. 6B illustrates a flowchart of an illustrative methodology for image analysis for functional and molecular cell profiling in accordance with one or more embodiments of the present disclosure.

In some embodiments, the image analysis engine 136 may include software functions to process images from the imaging device 121 and control the imagine device 121, including imaging device 121 calibration to capture images of cells in the filled well array 103, and to determine characteristics of the cells based on the images and changes through time. In some embodiments, to calibrate the imaging device 121, upon placing the filled well array 102 within the field of view 122 of the imaging device 121, the imaging device 121 may provide a feed of images of the filled well array 103 to the image analysis engine 136.

In some embodiments, at block 231, the image analysis engine 136 may check for array alignment. The orientation of cassette, slide, plate, petri dish or other element containing one or more array or other element may be very important. The boundary of the cassette, slide, plate, petri dish or other element containing one or more array or other element may need be aligned to make sure the array(s) of wells are straight or rotated in such a way as to allow for registration.

In some embodiments, at block 232, the image analysis engine 136 may calibrate an exposure time for the exposure of image capture by the imaging device 121. In some embodiments, based on the image intensity in the middle of chip, the device or user may need to adjust the best exposure time for each channel. For example, maximum exposure time accepted may be 100 msec and lower exposure time may be desirable as long as the signal is not too weak.

In some embodiments, at block 233, a first block or other location may be identified manually or automatically, where automatic selection is accomplished through the algorithm embedded in the acquisition software (as described above), and this may involves creating a grid over the array in a way that each zone in the grid specify the x and y location of each block.

Additionally, certain wells can be modified, such as by rotating square/rectangle wells by 45 degrees to assist in registration. (See pending application.).

In some embodiments, at block 234, the focusing may be adjusted manually or automatically, e.g., by the image analysis engine 136.

In some embodiments, at block 235, the image analysis engine 136 may initiate an imaging sequence. The number of frames and interval for imaging may be determined manually or automatically. The device may take images of different locations in sequence in order to capture images of every well or a portion of the wells at sufficient time points over a period of minutes, hours or days to facilitate analysis. For example, using a 20× objective, the device may image blocks of 36 wells with 50-micron dimensions (6 by 6 blocks) or blocks of 64 wells with 40-micron dimensions (8 by 8 blocks), for example imaging each block every five minutes over eight hours. Objectives with higher/lower magnification may be used for higher/lower resolution and lower/higher number of wells in each block. The imaging is done through intervals meaning that after a specified time point, the same blocks are imaged sequentially. This creates a time-lapse video of the blocks by arranging the images sequentially. For recognizing the features, the fluorescent intensity of cell labels and death marker is used. Also, the bright filed image can be used to identify several parameters such as morphology or cell-cell-interaction.

In some embodiments, the image analysis engine 136 may evaluate dynamic and/or functional performance of cells and cell-cell interactions, as well as the ability to enable the direct link of dynamic/functional and molecular profiles of individual cells at high throughput and in a distributed fashion. The devices also enable dynamic imaging of mitochondria and function of individual T cells and other cells, the image analysis engine 136 allows for automated and user-assisted image processing.

Accordingly, the resulting images 202 from the imaging sequence at block 235 may be provided to the computing device 240, such as, e.g., the control system 140 of the cellular profiling system 100 to control the sample retrieval stage G to select and retrieve a particular sample from the well array 102 using the sample picker 112.

In some embodiments, the computing device 240 may also or alternatively use the images 202 to evaluate different CAR designs for CAR T and other cell therapies to determine which candidates to move forward in development based on the mitochondria and function of individual cells.

In some embodiments, the computing device 240 may also or alternatively use the images 202 to predict response based on the functional performance (and possibly integrating other molecular profiling) of allogeneic immune cell products, antibodies, vaccine-exposed cells, target cell lines and/or patient-derived immune cells, disease cells and other cells, including before manufacturing, after manufacturing or after treatment, e.g., by preparing the samples with the allogeneic immune cell products, antibodies, vaccine-exposed cells, target cell lines and/or patient-derived immune cells, disease cells and other cells.

In some embodiments, to predict the response, timing of the capture of the images 202 via the imaging sequence 235 may characterize 100 seconds of parameters, such as, e.g., cell motility, time for cell to encounter the target, time of contact, morphology of cell and its target, the apoptosis pathways, secretion of multiple cytokines, antigen engulfment, etc. In some embodiments, algorithms for image analysis 236 are comprised of nanowell detection (e.g., using a local maximum clustering algorithm), nanowell localization (e.g., using normalized cross-correlation based template fitting, optionally with a Fourier implementation), pre-processing (background subtraction and correction for spectral overlap between emission spectra of multiple fluorescence dye), nanowell prioritization (wherein a subset of nanowells and/or blocks of nanowells are analyzed to determine which nanowells and/or blocks of nanowells should be imaged and/or analyzed in later steps, which may be based on an experiment design, which for example, may define), cell counting (normalized multi-threshold distance map (NMTDM) algorithm), cell tracking (e.g., a histogram-based cell count estimate to re-segment the cells de novo by a normalized spectral clustering of image pixels to detect cells of diverse shapes, and estimate clusters (cells) with similar sizes), and cell-cell contact algorithms.

In some embodiments, the computing device 240 may also or alternatively use the images 202 to evaluate the performance of manufactured products such as “release testing”.

FIG. 6C illustrates a flowchart of another illustrative methodology for image analysis for functional and molecular cell profiling in accordance with one or more embodiments of the present disclosure.

In some embodiments, the image analysis engine 136 may receive the images 202 of the filled well array 103 from the imaging device 121. In some embodiments, the image analysis engine 136 may implement image analysis algorithms and models to profile each nanowell of a nanowell filled well array 103.

In some embodiments, nanowell detection may be performed at block 331 on the images 302 to detect each nanowell in the filled well array 103. In some embodiments, nanowell detection may include using a local maximum clustering algorithm or other suitable detection model or any combination thereof.

In some embodiments, the image analysis engine 136 may perform nanowell localization at block 332. In some embodiments, nanowell localization may include e.g., using normalized cross-correlation based template fitting, optionally with a Fourier implementation, or other suitable localization model or any combination thereof.

In some embodiments, the image analysis engine 136 may perform pre-processing at block 333. (background subtraction and correction for spectral overlap between emission spectra of multiple fluorescence dye, or other suitable pre-processing model and/or algorithm or any combination thereof.

In some embodiments, the image analysis engine 136 may perform nanowell prioritization at block 334. In some embodiments, nanowell prioritization may include analyzing a subset of nanowells and/or blocks of nanowells to determine which nanowells and/or blocks of nanowells should be imaged and/or analyzed in later steps, which may be based on an experiment design.

In some embodiments, the image analysis engine 136 may then perform an initial cell counting and refinement process at block 335. In some embodiments, initial cell counting may include, e.g., constructing a histogram for each image and performing a histogram-based cell count estimate.

In some embodiments, the cells in each nanowell may be re-segmented according to cell re-segmentation at block 336. In some embodiments, re-segmentation may utilize the histogram-based cell count estimate to re-segment the cells de novo by a normalized spectral clustering of image pixels to detect cells of diverse shapes. In some embodiments, re-segmentation may include, e.g., normalized cross-correlation (NCC) for image registration, Fourier implementation to normalize in the spatial domain, local maximum clustering algorithm on the best-fitting NCC response to detect well centers, leveling to correct illumination variations by subtracting the local background estimated at each pixel using a Gaussian kernel, unmixing by a linear inverse method, image smoothing using a median filter (with radius rm˜=3) while preserving cell boundaries, cell body detection with a normalized multi-threshold distance map (NMTDM) that averages distance maps corresponding to multiple thresholds, local maxima clustering for cell detection, normalized spectral clustering of image pixels to re-segment cells.

In some embodiments, upon cell re-segmentation, the nanowells may be profiled according to cell features and cell behaviors. Accordingly, in some embodiments, features may be produced via feature computation at block 337a. In some embodiments, feature computations may include, for each cell, automated segmentation and tracking operations that produce multiple time series of primary features including, e.g., cell location, area, instantaneous speed, cell shape, and the contact measure. In addition, target cell death events (apoptosis) are detected using death marker fluorescence intensity, is measured as another primary feature.

In some embodiments, feature computation at block 337a may include computing cellular features at the scale of each nanowell, specifically, the number of effector cells, target cells, dead effectors, contacted targets, and killed targets, among other cellular features. In some embodiments, features such cell counts of various types of cells may include, e.g., employing a normalized multi-threshold distance map (NMTDM) algorithm to identified cells.

In some embodiments, feature computation at block 337a may include computing a set of secondary features for each cell. For each cell, secondary features may be produced such as the average speed prior to first contact, average speed during the contact phase, average cell eccentricity prior to first contact, average eccentricity during the contact phase, time elapsed between first contact and death, total contact duration between first contact and death, time duration before first contact, the number of conjugations prior to target cell death.

In some embodiments, cell tracking at block 337b may be performed to track the movement and behavior of each cell or aggregates of cells or both. In some embodiments, the cell tracking 337b may including tracking or calculating changes across images in the normalized spectral clustering from the cell re-segmentation of block 336. For example, cell tracking at block 337b may include, e.g., a directed graph for confinement-constrained cell tracking.

In some embodiments, cell behavior may also include cell to cell (“cell-cell”) contact in each nanowell. In some embodiments, a cell-cell contact analysis at block 337c is performed using suitable cell-cell contact algorithms. For example, the cell-cell contact algorithms may include, e.g., soft cell interaction measure CI for quantifying the interaction of a cell with its surrounding cells, or a regional convolutional neural network for cellular and subcellular detection and segmentation, or other cell-cell algorithm or any combination thereof. In some embodiments, the cell-cell contact analysis of block 337c may include determining, e.g., characterizing timer periods (e.g., 30 seconds, 60 seconds, 100 seconds, 200 seconds, etc.) of parameters, such as, e.g., cell motility, time for cell to encounter the target, time of contact, morphology of cell and its target, the apoptosis pathways, secretion of multiple cytokines, antigen engulfment, etc.

In some embodiments, features, cell tracking results and cell-cell contact results may be aggregated and analyzed in a profiling and data analysis at block 338. In some embodiments, the profiling and data analysis may include statistical and other analyses of the behavior of cells in each nanowell through time to characterize the behavior and traits of each detected cell type. The results of the profiling and data analysis at block 338 may then be provided to a user via the computing device 240.

FIG. 7 depicts a block diagram of an exemplary computer-based system and platform 700 in accordance with one or more embodiments of the present disclosure. However, not all of these components may be required to practice one or more embodiments, and variations in the arrangement and type of the components may be made without departing from the spirit or scope of various embodiments of the present disclosure. In some embodiments, the illustrative computing devices and the illustrative computing components of the exemplary computer-based system and platform 700 may be configured to manage a large number of members and concurrent transactions, as detailed herein. In some embodiments, the exemplary computer-based system and platform 700 may be based on a scalable computer and network architecture that incorporates varies strategies for assessing the data, caching, searching, and/or database connection pooling. An example of the scalable architecture is an architecture that is capable of operating multiple servers.

In some embodiments, referring to FIG. 7, member computing device 702, member computing device 703 through member computing device 704 (e.g., clients) of the exemplary computer-based system and platform 700 may include virtually any computing device capable of receiving and sending a message over a network (e.g., cloud network), such as network 705, to and from another computing device, such as servers 706 and 707, each other, and the like. In some embodiments, the member devices 702-704 may be personal computers, multiprocessor systems, microprocessor-based or programmable consumer electronics, network PCs, and the like. In some embodiments, one or more member devices within member devices 702-704 may include computing devices that typically connect using a wireless communications medium such as cell phones, smart phones, pagers, walkie talkies, radio frequency (RF) devices, infrared (IR) devices, citizens band radio, integrated devices combining one or more of the preceding devices, or virtually any mobile computing device, and the like. In some embodiments, one or more member devices within member devices 702-704 may be devices that are capable of connecting using a wired or wireless communication medium such as a PDA, POCKET PC, wearable computer, a laptop, tablet, desktop computer, a netbook, a video game device, a pager, a smart phone, an ultra-mobile personal computer (UMPC), and/or any other device that is equipped to communicate over a wired and/or wireless communication medium (e.g., NFC, RFID, NBIOT, 3G, 4G, 5G, GSM, GPRS, WiFi, WiMax, CDMA, OFDM, OFDMA, LTE, satellite, ZigBee, etc.). In some embodiments, one or more member devices within member devices 702-704 may include may run one or more applications, such as Internet browsers, mobile applications, voice calls, video games, videoconferencing, and email, among others. In some embodiments, one or more member devices within member devices 702-704 may be configured to receive and to send web pages, and the like. In some embodiments, an exemplary specifically programmed browser application of the present disclosure may be configured to receive and display graphics, text, multimedia, and the like, employing virtually any web based language, including, but not limited to Standard Generalized Markup Language (SMGL), such as HyperText Markup Language (HTML), a wireless application protocol (WAP), a Handheld Device Markup Language (HDML), such as Wireless Markup Language (WML), WMLScript, XML, JavaScript, and the like. In some embodiments, a member device within member devices 702-704 may be specifically programmed by either Java, .Net, QT, C, C++, Python, PHP and/or other suitable programming language. In some embodiment of the device software, device control may be distributed between multiple standalone applications. In some embodiments, software components/applications can be updated and redeployed remotely as individual units or as a full software suite. In some embodiments, a member device may periodically report status or send alerts over text or email. In some embodiments, a member device may contain a data recorder which is remotely downloadable by the user using network protocols such as FTP, SSH, or other file transfer mechanisms. In some embodiments, a member device may provide several levels of user interface, for example, advance user, standard user. In some embodiments, one or more member devices within member devices 702-704 may be specifically programmed include or execute an application to perform a variety of possible tasks, such as, without limitation, messaging functionality, browsing, searching, playing, streaming or displaying various forms of content, including locally stored or uploaded messages, images and/or video, and/or games.

In some embodiments, the exemplary network 705 may provide network access, data transport and/or other services to any computing device coupled to it. In some embodiments, the exemplary network 705 may include and implement at least one specialized network architecture that may be based at least in part on one or more standards set by, for example, without limitation, Global System for Mobile communication (GSM) Association, the Internet Engineering Task Force (IETF), and the Worldwide Interoperability for Microwave Access (WiMAX) forum. In some embodiments, the exemplary network 705 may implement one or more of a GSM architecture, a General Packet Radio Service (GPRS) architecture, a Universal Mobile Telecommunications System (UMTS) architecture, and an evolution of UMTS referred to as Long Term Evolution (LTE). In some embodiments, the exemplary network 705 may include and implement, as an alternative or in conjunction with one or more of the above, a WiMAX architecture defined by the WiMAX forum. In some embodiments and, optionally, in combination of any embodiment described above or below, the exemplary network 705 may also include, for instance, at least one of a local area network (LAN), a wide area network (WAN), the Internet, a virtual LAN (VLAN), an enterprise LAN, a layer 3 virtual private network (VPN), an enterprise IP network, or any combination thereof. In some embodiments and, optionally, in combination of any embodiment described above or below, at least one computer network communication over the exemplary network 705 may be transmitted based at least in part on one of more communication modes such as but not limited to: NFC, RFID, Narrow Band Internet of Things (NBIOT), ZigBee, 3G, 4G, 5G, GSM, GPRS, WiFi, WiMax, CDMA, OFDM, OFDMA, LTE, satellite and any combination thereof. In some embodiments, the exemplary network 705 may also include mass storage, such as network attached storage (NAS), a storage area network (SAN), a content delivery network (CDN) or other forms of computer or machine readable media.

In some embodiments, the exemplary server 706 or the exemplary server 707 may be a web server (or a series of servers) running a network operating system, examples of which may include but are not limited to Apache on Linux or Microsoft IIS (Internet Information Services). In some embodiments, the exemplary server 706 or the exemplary server 707 may be used for and/or provide cloud and/or network computing. Although not shown in FIG. 7, in some embodiments, the exemplary server 706 or the exemplary server 707 may have connections to external systems like email, SMS messaging, text messaging, ad content providers, etc. Any of the features of the exemplary server 706 may be also implemented in the exemplary server 707 and vice versa.

In some embodiments, one or more of the exemplary servers 706 and 707 may be specifically programmed to perform, in non-limiting example, as authentication servers, search servers, email servers, social networking services servers, Short Message Service (SMS) servers, Instant Messaging (IM) servers, Multimedia Messaging Service (MMS) servers, exchange servers, photo-sharing services servers, advertisement providing servers, financial/banking-related services servers, travel services servers, or any similarly suitable service-base servers for users of the member computing devices 701-704.

In some embodiments and, optionally, in combination of any embodiment described above or below, for example, one or more exemplary computing member devices 702-704, the exemplary server 706, and/or the exemplary server 707 may include a specifically programmed software module that may be configured to send, process, and receive information using a scripting language, a remote procedure call, an email, a tweet, Short Message Service (SMS), Multimedia Message Service (MMS), instant messaging (IM), an application programming interface, Simple Object Access Protocol (SOAP) methods, Common Object Request Broker Architecture (CORBA), HTTP (Hypertext Transfer Protocol), REST (Representational State Transfer), SOAP (Simple Object Transfer Protocol), MLLP (Minimum Lower Layer Protocol), or any combination thereof.

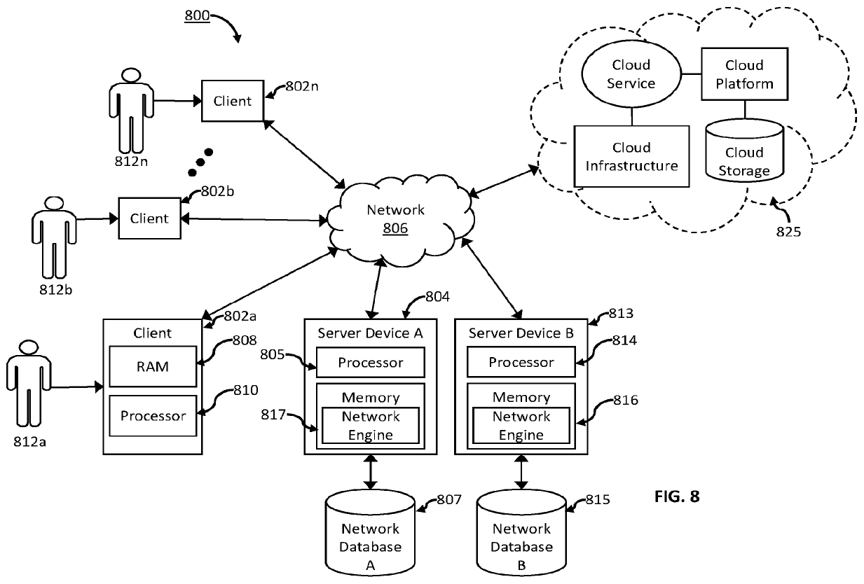

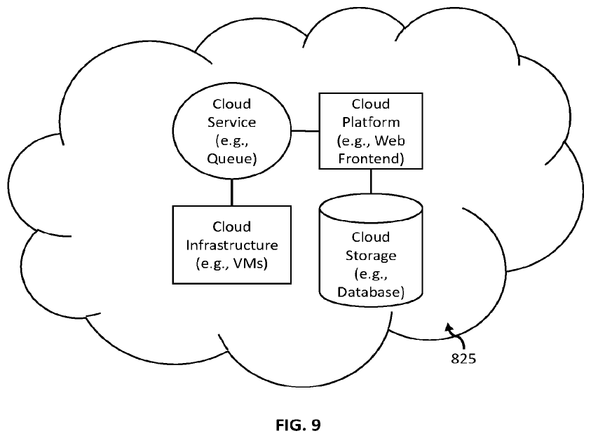

FIG. 8 depicts a block diagram of another exemplary computer-based system and platform 800 in accordance with one or more embodiments of the present disclosure. However, not all of these components may be required to practice one or more embodiments, and variations in the arrangement and type of the components may be made without departing from the spirit or scope of various embodiments of the present disclosure. In some embodiments, the member computing device 802a, member computing device 802b through member computing device 802n shown each at least includes a computer-readable medium, such as a random-access memory (RAM) 808 coupled to a processor 810 or FLASH memory. In some embodiments, the processor 810 may execute computer-executable program instructions stored in memory 808. In some embodiments, the processor 810 may include a microprocessor, an ASIC, and/or a state machine. In some embodiments, the processor 810 may include, or may be in communication with, media, for example computer-readable media, which stores instructions that, when executed by the processor 810, may cause the processor 810 to perform one or more steps described herein. In some embodiments, examples of computer-readable media may include, but are not limited to, an electronic, optical, magnetic, or other storage or transmission device capable of providing a processor, such as the processor 810 of client 802a, with computer-readable instructions. In some embodiments, other examples of suitable media may include, but are not limited to, a floppy disk, CD-ROM, DVD, magnetic disk, memory chip, ROM, RAM, an ASIC, a configured processor, all optical media, all magnetic tape or other magnetic media, or any other medium from which a computer processor can read instructions. Also, various other forms of computer-readable media may transmit or carry instructions to a computer, including a router, private or public network, or other transmission device or channel, both wired and wireless. In some embodiments, the instructions may comprise code from any computer-programming language, including, for example, C, C++, Visual Basic, Java, Python, Perl, JavaScript, and etc.

In some embodiments, member computing devices 802a through 802n may also comprise a number of external or internal devices such as a mouse, a CD-ROM, DVD, a physical or virtual keyboard, a display, or other input or output devices. In some embodiments, examples of member computing devices 802a through 802n (e.g., clients) may be any type of processor-based platforms that are connected to a network 806 such as, without limitation, personal computers, digital assistants, personal digital assistants, smart phones, pagers, digital tablets, laptop computers, Internet appliances, and other processor-based devices. In some embodiments, member computing devices 802a through 802n may be specifically programmed with one or more application programs in accordance with one or more principles/methodologies detailed herein. In some embodiments, member computing devices 802a through 802n may operate on any operating system capable of supporting a browser or browser-enabled application, such as Microsoft™, Windows™, and/or Linux. In some embodiments, member computing devices 802a through 802n shown may include, for example, personal computers executing a browser application program such as Microsoft Corporation's Internet Explorer™, Apple Computer, Inc.'s Safari™, Mozilla Firefox, and/or Opera. In some embodiments, through the member computing client devices 802a through 802n, user 812a, user 812b through user 812n, may communicate over the exemplary network 806 with each other and/or with other systems and/or devices coupled to the network 806. As shown in FIG. 8, exemplary server devices 804 and 813 may include processor 805 and processor 814, respectively, as well as memory 817 and memory 816, respectively. In some embodiments, the server devices 804 and 813 may be also coupled to the network 806. In some embodiments, one or more member computing devices 802a through 802n may be mobile clients.

In some embodiments, at least one database of exemplary databases 807 and 815 may be any type of database, including a database managed by a database management system (DBMS). In some embodiments, an exemplary DBMS-managed database may be specifically programmed as an engine that controls organization, storage, management, and/or retrieval of data in the respective database. In some embodiments, the exemplary DBMS-managed database may be specifically programmed to provide the ability to query, backup and replicate, enforce rules, provide security, compute, perform change and access logging, and/or automate optimization. In some embodiments, the exemplary DBMS-managed database may be chosen from Oracle database, IBM DB2, Adaptive Server Enterprise, FileMaker, Microsoft Access, Microsoft SQL Server, MySQL, PostgreSQL, and a NoSQL implementation. In some embodiments, the exemplary DBMS-managed database may be specifically programmed to define each respective schema of each database in the exemplary DBMS, according to a particular database model of the present disclosure which may include a hierarchical model, network model, relational model, object model, or some other suitable organization that may result in one or more applicable data structures that may include fields, records, files, and/or objects. In some embodiments, the exemplary DBMS-managed database may be specifically programmed to include metadata about the data that is stored.

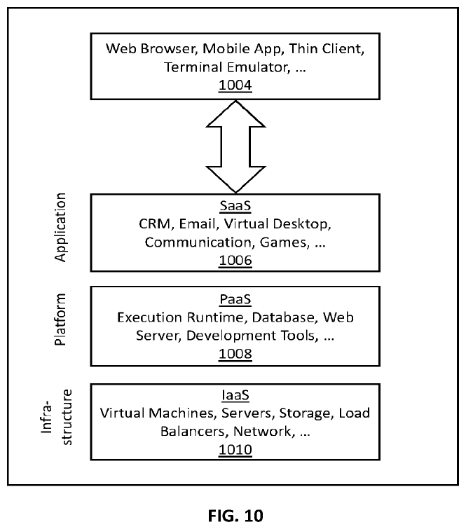

In some embodiments, the exemplary inventive computer-based systems/platforms, the exemplary inventive computer-based devices, and/or the exemplary inventive computer-based components of the present disclosure may be specifically configured to operate in a cloud computing/architecture 825 such as, but not limiting to: infrastructure a service (IaaS) 1010, platform as a service (PaaS) 1008, and/or software as a service (SaaS) 1006 using a web browser, mobile app, thin client, terminal emulator or other endpoint 1004. FIGS. 9 and 10 illustrate schematics of exemplary implementations of the cloud computing/architecture(s) in which the exemplary inventive computer-based systems/platforms, the exemplary inventive computer-based devices, and/or the exemplary inventive computer-based components of the present disclosure may be specifically configured to operate.

FIGS. 11A, 11B and 11C illustrate example screenshots of the image analysis (e.g., as displayed on the computing device 240) in accordance with aspects of embodiments of the present invention.

FIG. 11A illustrates a nanowell profiler window displays a table of single-cell measurements with each row linked to a specific nanowell video in accordance with aspects of embodiments of the present invention.

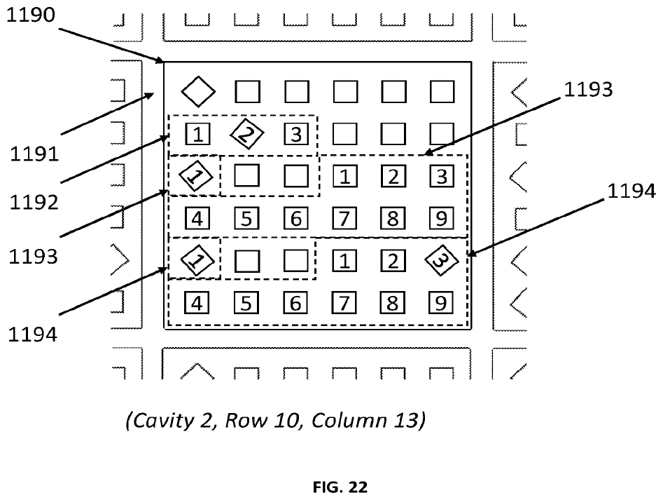

FIG. 111B, illustrates video display and editing window for the selected nanowell showing the contents of this nanowell with segmentation masks overlaid in accordance with aspects of embodiments of the present invention. In some embodiments, clicking the green button displays the detailed time-series measurements for this nanowell. In some embodiments, clicking the red button initializes the nanowell editor allows efficient correction of automated segmentation or tracking errors.

FIG. 11C illustrates a nanowell editor to allow for correction of the automated segmentation tracking errors in accordance with aspects of embodiments of the present invention.

In some embodiments, the user interface enables setting parameters for desired volumes to be loaded onto one or more array and/or cassette, slide, plate, petri dish or other element containing one or more array or other element on a stage or other element to hold the cassette, slide, plate, petri dish or other element containing one or more array or other element.

In some embodiments, the user interface enables setting experiment parameters such as time between image capture, total time and/or start and end times for experiments.

In some embodiments, the user interface enables initiating experiments.

In some embodiments, the user interface enables setting analysis parameters such as thresholds for determining cell death.

In some embodiments, the user interface enables outputting data such as tables, statistics, plots, graphs, images and/or videos to a screen, disk, local network, internet or other connection.

Embodiments of the user interface may include a keyboard, keypad, mouse, track pad or remote connection to an application on another device or on the web.

### Examples of Cellular Profiling System

In FIG. 12, a system 1100 according to one or more embodiments of the present disclosure is shown. In some embodiments, the cellular profiling system 100 above may include system 1100, which may be contained in an enclosure 1104. System 1100 may include a door to enable loading and removing one or more arrays, samples, reagents, and/or pipette tips. System 1100 may include a controller 1102 to view and/or modify settings and measurements related to elements such as imaging frequency, experiment duration, temperature, humidity and/or CO2. The enclosure 1104 is fastened with fasteners 1103.

In FIG. 13, a system 1110 according to one or more embodiments of the present disclosure is shown in a cutaway view from the front. In some embodiments, the cellular profiling system 100 above may include the system 1110, which may include the enclosure 1104 held together with fasteners 1101. The system 1110 may include a shuttle and scanning stage 1111 to move an array into an incubator 1116 and to move the array so it the system can image different portions of the array, a fluorescence source 1112, electronics 1113, a microscope assembly 1114, elements for condensation prevention 1115 to prevent condensation on a window into the incubator 1116, and an incubator controller 1117 to control humidity and/or CO2.

In FIG. 14, a system/device 1120 including an array carrier 1120 is shown according to one or more embodiments of the present disclosure. In some embodiments, the cellular profiling system 100 above may include the system/device 1120. The array carrier may include a lid, e.g., with edges 1121 to hold an array, and holes and slots for placement of the carrier in the system 1122. The edges 1121 may be chamfered, beveled or otherwise shaped to receive and hold the array and/or carrier.

In FIG. 15, a system/device 1130 including an array carrier 1120 is shown according to one or more embodiments of the present disclosure. In some embodiments, the cellular profiling system 100 above may include the system/device 1130. The array carrier may include a lid with chamfered edges 1121 to hold an array 1135 and holes and slots for placement in the system 1122. The carrier may include a finger gap 1133 for lifting the lid 1121 and magnets to hold the lid in the down position 1134.

In FIG. 16, a system/device 1140 including a microscope assembly 1130 is shown according to one or more embodiments of the present disclosure. The microscope assembly 1140 may include a white light illuminator 1141, a blocking filter 1142, a condenser 1143, an objective 1144, a light guide 1145, a beam splitter 1146, a tube lens 1147, a fold mirror 1148, and a camera 1149.

In FIG. 17, a portion 1150 of a system that provides condensation prevention is shown according to one or more embodiments of the present disclosure, including a fan heater envelope 1151 for holding a fan heater that blows air through a duct 1153; the top of an incubator body 1152, which provides a contained space for maintaining air with certain humidity and/or temperature conditions and contains an incubator window 1154, which allows viewing the array from above, and inputs for CO2 and humidity 1155.

In FIG. 18, a system 1160 is shown in cutaway view from the top, in accordance with one or more embodiments of the present disclosure. The system 1160 may include a pipette tip rack 1161 for holding pipette tips to be accessed by a pipettor (not shown), a liquid and solid waste reservoir 1162 where waste is deposited by a pipettor (not shown), an input rack 1163 which holds tubes of cells, reagents, and other inputs to be accessed by a pipettor (not shown), a loading station 1164 which may include an array carrier 1165 and a shuttle 1166 to enable the array to be moved into and out of the imaging station 1167 and to be moved within the imaging station 1167 to enable imaging different portions of the array, an imaging station 1167 which may include a microscope assembly containing a white light illuminator, a blocking filter, a condenser, an objective, a light guide, a beam splitter, a tube lens, a fold mirror, a camera, and an incubator contained in an incubator body 1169, which may include an incubator window 1168.

In FIG. 19, a system 1170 is shown in cutaway view from the top according to one or more embodiments of the present disclosure. System 1170 may include at least one embodiment where sample loading is not used. The system 1170 may include a loading station 1174 which may include an array carrier 1175 and a shuttle 1176 to enable the array to be moved into and out of the incubator and to be moved to enable imaging different portions of the array, an imaging station 1177 which may include a microscope assembly containing a white light illuminator, a blocking filter, a condenser, an objective, a light guide, a beam splitter, a tube lens, a fold mirror, a camera, and an incubator contained in an incubator body 1179, which may include an incubator window 1178.

In FIG. 20, a device 1180 is shown is shown according to one or more embodiments of the present disclosure. In some embodiments, the cellular profiling system 100 above may interface with the array device 1180 by utilizing the array carrier 1120 or another element of the profiling system 100. In some embodiments, the array device may include bottom structure 1181 that has a thickness sufficient to provide structural support for the device 1180, e.g., greater than 0.5 mm, or other suitable thickness such as in a range of 1 mm to 2 mm. In some embodiments, the bottom structure 1181 may also accommodate a thinner bottom 1182 of each cavity 1189 the structure 1184, where the thinner bottom 1182 has a thickness to enable imaging of one of more arrays of wells, such as, e.g., a thickness in a range of about 100 microns to 1000 microns, or any thickness less than or equal to about 500 microns, or other suitable thickness. One or more corners 1183 may be chamfered, beveled or otherwise shaped to enable the system 100 to receive and hold the device. In some embodiments, the array may have the structure 1184 that provides one or more cavities 1189 that can hold media or other liquid, each of which may have an array of wells. The device 1180 may be a cassette, slide, plate, petri dish or other format. In some embodiments, the wall of the cavity 1189 has one or more “V” shapes to enable removal of liquid from the cavity during sample loading or at other times.

In FIG. 21, a device 1180 is shown in cutaway view from the side according to one or more embodiments of the present disclosure. In some embodiments, the array device 1180 may include bottom structure 1181 that has a thickness sufficient to provide structural support for the device 1180, e.g., greater than 0.5 mm, or other suitable thickness such as in a range of 1 mm to 2 mm. In some embodiments, the bottom structure 1181 may also accommodate a thinner bottom 1182 of each cavity 1189 the structure 1184, where the thinner bottom 1182 has a thickness to enable imaging of one of more arrays of wells, such as, e.g., a thickness in a range of about 100 microns to 1000 microns, or any thickness less than or equal to about 500 microns, or other suitable thickness to enable imaging of one of more array of wells 1185 with objectives that have a numerical aperture of at least 0.6. One or more corners 1183 may be chamfered, beveled or otherwise shaped to enable the system 100 to receive and hold the device. In some embodiments, the array may have a structure that provides one or more cavities 1189 that can hold media or other liquid 1186, each of which may have an array of wells 1185. The device 1180 may be a cassette, slide, plate, petri dish or other format. In some embodiments, the thinner bottom 1182 has consistent flatness to reduce the need to repeatedly focus imaging components, which in some embodiments may include any suitable variation in thickness, such as less than 20 microns. In some embodiments, the consistency of the position in the Z direction of the bottom of any individual well, or of a sufficient number of individual wells to generate data from a majority of wells, in the array of wells 1185 is sufficiently consistent to reduce the need to repeatedly focus imaging components, which in some embodiments may include any consistency, e.g., less than 30 microns, less than 25 microns, less than 20 microns, less than 15 microns, less than 10 microns, less than 5 microns, etc.

In FIG. 22, a single block of wells 1190 from an array of wells 1185 is shown from above according to one or more embodiments of the present disclosure (with neighboring blocks partially shown). In some embodiments, the block of wells contains wells with depth, width and length of 10- to 200-microns or other suitable dimensions for maximizing a number of samples via imaging. In some embodiments, the number of wells per block corresponds to the field of view of the camera included in the system. In some embodiments, the array of wells 1185 may include a channel 1191 around or next to the block of wells 1190, where in some embodiments the width and depth of the channel is similar to the width and depth of individual wells, and in some embodiments the channel may be in only one or more than one direction. In some embodiments, the array of wells 1185 is made of a material that is optically transparent in the UV and IR regions. In some embodiments, the block of wells 1190 is made of a material that is optically transparent in the UV and IR regions. In some embodiments, individual wells of a group of wells 1192 are rotated in a fashion that indicates the position of the group of blocks of wells in the cavity 1184. In some embodiments, individual wells of a group of wells 1193 are rotated in a fashion that indicates the row position of the block of wells 1190 on an array of wells 1185. In some embodiments, individual wells of a group of wells 1194 are rotated in a fashion that indicates the column position of the block of wells 1190 on an array of wells 1185. In some embodiments, the distance from the edge of the wall of the cavity 1184 or material supporting the wall of the cavity 1181 sufficient to enable imaging of all wells in each block of wells 1190, such as, e.g., at least 2 mm, 3 mm, 4 mm, or more.

It is understood that at least one aspect/functionality of various embodiments described herein can be performed in real-time and/or dynamically. As used herein, the term “real-time” is directed to an event/action that can occur instantaneously or almost instantaneously in time when another event/action has occurred. For example, the “real-time processing,” “real-time computation,” and “real-time execution” all pertain to the performance of a computation during the actual time that the related physical process (e.g., a user interacting with an application on a mobile device) occurs, in order that results of the computation can be used in guiding the physical process.

As used herein, the term “dynamically” and term “automatically,” and their logical and/or linguistic relatives and/or derivatives, mean that certain events and/or actions can be triggered and/or occur without any human intervention. In some embodiments, events and/or actions in accordance with the present disclosure can be in real-time and/or based on a predetermined periodicity of at least one of: nanosecond, several nanoseconds, millisecond, several milliseconds, second, several seconds, minute, several minutes, hourly, several hours, daily, several days, weekly, monthly, etc.

As used herein, the term “runtime” corresponds to any behavior that is dynamically determined during an execution of a software application or at least a portion of software application.

In some embodiments, exemplary inventive, specially programmed computing systems and platforms with associated devices are configured to operate in the distributed network environment, communicating with one another over one or more suitable data communication networks (e.g., the Internet, satellite, etc.) and utilizing one or more suitable data communication protocols/modes such as, without limitation, IPX/SPX, X.25, AX.25, AppleTalk™, TCP/IP (e.g., HTTP), near-field wireless communication (NFC), RFID, Narrow Band Internet of Things (NBIOT), 3G, 4G, 5G, GSM, GPRS, WiFi, WiMax, CDMA, satellite, ZigBee, and other suitable communication modes.

In some embodiments, the NFC can represent a short-range wireless communications technology in which NFC-enabled devices are “swiped,” “bumped,” “tap” or otherwise moved in close proximity to communicate. In some embodiments, the NFC could include a set of short-range wireless technologies, typically requiring a distance of 10 cm or less. In some embodiments, the NFC may operate at 13.56 MHz on ISO/IEC 18000-3 air interface and at rates ranging from 106 kbit/s to 424 kbit/s. In some embodiments, the NFC can involve an initiator and a target; the initiator actively generates an RF field that can power a passive target. In some embodiment, this can enable NFC targets to take very simple form factors such as tags, stickers, key fobs, or cards that do not require batteries. In some embodiments, the NFC's peer-to-peer communication can be conducted when a plurality of NFC-enable devices (e.g., smartphones) within close proximity of each other.

In some embodiments, materials used for arrays of wells and supporting materials may include one or more of glass, polydimethylsiloxane (PDMS or dimethicone), cyclic olefin copolymer (COC), cyclic olefin polymer (COP), UV stabilized resin, or other materials with refractive indices that are compatible with imaging with objectives that have a numerical aperture of at least 0.6 (or other suitable numerical aperture, such as 0.4, 0.5, 0.6, 0.7, 0.8, 0.9, 1.0, etc.), and/or auto-fluorescence sufficient for imaging with objectives that have a numerical aperture of at least 0.6 (or other suitable numerical aperture, such as 0.4, 0.5, 0.6, 0.7, 0.8, 0.9, 1.0, etc.) and/or an elastic modulus that enables contact from a caliper made of glass or other material without resulting in damaging the glass or other material. In some embodiments, materials used for cavities to contain media or other liquid are sufficient to enable bonding to other surfaces such that liquids do not leak under incubation conditions consistent with 37 C for up to 72 hours.

The material disclosed herein may be implemented in software or firmware or a combination of them or as instructions stored on a machine-readable medium, which may be read and executed by one or more processors. A machine-readable medium may include any medium and/or mechanism for storing or transmitting information in a form readable by a machine (e.g., a computing device). For example, a machine-readable medium may include read only memory (ROM); random access memory (RAM); magnetic disk storage media; optical storage media; flash memory devices; electrical, optical, acoustical or other forms of propagated signals (e.g., carrier waves, infrared signals, digital signals, etc.), and others.

As used herein, the terms “computer engine” and “engine” identify at least one software component and/or a combination of at least one software component and at least one hardware component which are designed/programmed/configured to manage/control other software and/or hardware components (such as the libraries, software development kits (SDKs), objects, etc.).

Examples of hardware elements may include processors, microprocessors, circuits, circuit elements (e.g., transistors, resistors, capacitors, inductors, and so forth), integrated circuits, application specific integrated circuits (ASIC), programmable logic devices (PLD), digital signal processors (DSP), field programmable gate array (FPGA), logic gates, registers, semiconductor device, chips, microchips, chip sets, and so forth. In some embodiments, the one or more processors may be implemented as a Complex Instruction Set Computer (CISC) or Reduced Instruction Set Computer (RISC) processors; x86 instruction set compatible processors, multi-core, or any other microprocessor or central processing unit (CPU), graphical processing unit (GPU), neural processing unit (NPU), etc. In various implementations, the one or more processors may be dual-core processor(s), dual-core mobile processor(s), and so forth. In some embodiments, the hardware elements may include peripheral hardware for supporting the hardware elements, such as, e.g., cooling devices (air and/or liquid cooling, radiator, heat sink, etc.), power supply, uninterrupted power supply, memory devices such as random access memory (RAM), input/output (I/O) interfaces, etc.

Computer-related systems, computer systems, and systems, as used herein, include any combination of hardware and software. Examples of software may include software components, programs, applications, operating system software, middleware, firmware, software modules, routines, subroutines, functions, methods, procedures, software interfaces, application program interfaces (API), instruction sets, computer code, computer code segments, words, values, symbols, or any combination thereof. Determining whether an embodiment is implemented using hardware elements and/or software elements may vary in accordance with any number of factors, such as desired computational rate, power levels, heat tolerances, processing cycle budget, input data rates, output data rates, memory resources, data bus speeds and other design or performance constraints.

One or more aspects of at least one embodiment may be implemented by representative instructions stored on a machine-readable medium which represents various logic within the processor, which when read by a machine causes the machine to fabricate logic to perform the techniques described herein. Such representations, known as “IP cores” may be stored on a tangible, machine readable medium and supplied to various customers or manufacturing facilities to load into the fabrication machines that make the logic or processor. Of note, various embodiments described herein may, of course, be implemented using any appropriate hardware and/or computing software languages (e.g., C++, Objective-C, Swift, Java, JavaScript, Python, Perl, QT, etc.).

In some embodiments, one or more of illustrative computer-based systems or platforms of the present disclosure may include or be incorporated, partially or entirely into at least one personal computer (PC), laptop computer, ultra-laptop computer, tablet, touch pad, portable computer, handheld computer, palmtop computer, personal digital assistant (PDA), cellular telephone, combination cellular telephone/PDA, television, smart device (e.g., smart phone, smart tablet or smart television), mobile internet device (MID), messaging device, data communication device, and so forth.

As used herein, term “server” should be understood to refer to a service point which provides processing, database, and communication facilities. By way of example, and not limitation, the term “server” can refer to a single, physical processor with associated communications and data storage and database facilities, or it can refer to a networked or clustered complex of processors and associated network and storage devices, as well as operating software and one or more database systems and application software that support the services provided by the server. Cloud servers are examples.

In some embodiments, as detailed herein, one or more of the computer-based systems of the present disclosure may obtain, manipulate, transfer, store, transform, generate, and/or output any digital object and/or data unit (e.g., from inside and/or outside of a particular application) that can be in any suitable form such as, without limitation, a file, a contact, a task, an email, a message, a map, an entire application (e.g., a calculator), data points, and other suitable data. In some embodiments, as detailed herein, one or more of the computer-based systems of the present disclosure may be implemented across one or more of various computer platforms such as, but not limited to: (1) FreeBSD, NetBSD, OpenBSD; (2) Linux (e.g., Debian, Ubuntu, Fedora, OpenSUSE, etc.); (3) Microsoft Windows™; (4) OpenVMS™; (5) OS X (MacOS™); (6) UNIX™; (7) Android; (8) iOS™; (9) Embedded Linux; (10) Tizen™; (11) WebOS™; (12) Adobe AIR™; (13) Binary Runtime Environment for Wireless (BREW™); (14) Cocoa™ (API); (15) Cocoa™ Touch; (16) Java™ Platforms; (17) JavaFX™; (18) QNX™; (19) Mono; (20) Google Blink; (21) Apple WebKit; (22) Mozilla Gecko™; (23) Mozilla XUL; (24) .NET Framework; (25) Silverlight™; (26) Open Web Platform; (27) Oracle Database; (28) Qt™; (29) SAP NetWeaver™; (30) Smartface™; (31) Vexi™; (32) Kubemetes™, (33) Docker, (34) Caffe and (35) Windows Runtime (WinRT™) or other suitable computer platforms or any combination thereof. In some embodiments, illustrative computer-based systems or platforms of the present disclosure may be configured to utilize hardwired circuitry that may be used in place of or in combination with software instructions to implement features consistent with principles of the disclosure. Thus, implementations consistent with principles of the disclosure are not limited to any specific combination of hardware circuitry and software. For example, various embodiments may be embodied in many different ways as a software component such as, without limitation, a stand-alone software package, a combination of software packages, or it may be a software package incorporated as a “tool” in a larger software product.

For example, exemplary software specifically programmed in accordance with one or more principles of the present disclosure may be downloadable from a network, for example, a website, as a stand-alone product or as an add-in package for installation in an existing software application. For example, exemplary software specifically programmed in accordance with one or more principles of the present disclosure may also be available as a client-server software application, or as a web-enabled software application. For example, exemplary software specifically programmed in accordance with one or more principles of the present disclosure may also be embodied as a software package installed on a hardware device.

In some embodiments, illustrative computer-based systems or platforms of the present disclosure may be configured to handle numerous concurrent users that may be, but is not limited to, at least 100 (e.g., but not limited to, 100-999), at least 1,000 (e.g., but not limited to, 1,000-9,999), at least 10,000 (e.g., but not limited to, 10,000-99,999), at least 100,000 (e.g., but not limited to, 100,000-999,999), at least 1,000,000 (e.g., but not limited to, 1,000,000-9,999,999), at least 10,000,000 (e.g., but not limited to, 10,000,000-99,999,999), at least 100,000,000 (e.g., but not limited to, 100,000,000-999,999,999), at least 1,000,000,000 (e.g., but not limited to, 1,000,000,000-999,999,999,999), and so on.

In some embodiments, illustrative computer-based systems or platforms of the present disclosure may be configured to output to distinct, specifically programmed graphical user interface implementations of the present disclosure (e.g., a desktop, a web app., etc.). In various implementations of the present disclosure, a final output may be displayed on a displaying screen which may be, without limitation, a screen of a computer, a screen of a mobile device, or the like. In various implementations, the display may be a holographic display. In various implementations, the display may be a transparent surface that may receive a visual projection. Such projections may convey various forms of information, images, or objects. For example, such projections may be a visual overlay for a mobile augmented reality (MAR) application.

In some embodiments, illustrative computer-based systems or platforms of the present disclosure may be configured to be utilized in various applications which may include, but not limited to, gaming, mobile-device games, video chats, video conferences, live video streaming, video streaming and/or augmented reality applications, mobile-device messenger applications, and others similarly suitable computer-device applications.

As used herein, the term “mobile electronic device,” or the like, may refer to any portable electronic device that may or may not be enabled with location tracking functionality (e.g., MAC address, Internet Protocol (IP) address, or the like). For example, a mobile electronic device can include, but is not limited to, a mobile phone, Personal Digital Assistant (PDA), Blackberry™, Pager, Smartphone, or any other reasonable mobile electronic device.

As used herein, terms “proximity detection,” “locating,” “location data,” “location information,” and “location tracking” refer to any form of location tracking technology or locating method that can be used to provide a location of, for example, a particular computing device, system or platform of the present disclosure and any associated computing devices, based at least in part on one or more of the following techniques and devices, without limitation: accelerometer(s), gyroscope(s), Global Positioning Systems (GPS); GPS accessed using Bluetooth™; GPS accessed using any reasonable form of wireless and non-wireless communication; WiFi™ server location data; Bluetooth™ based location data; triangulation such as, but not limited to, network based triangulation, WiFi™ server information based triangulation, Bluetooth™ server information based triangulation; Cell Identification based triangulation, Enhanced Cell Identification based triangulation, Uplink-Time difference of arrival (U-TDOA) based triangulation, Time of arrival (TOA) based triangulation, Angle of arrival (AOA) based triangulation; techniques and systems using a geographic coordinate system such as, but not limited to, longitudinal and latitudinal based, geodesic height based, Cartesian coordinates based; Radio Frequency Identification such as, but not limited to, Long range RFID, Short range RFID; using any form of RFID tag such as, but not limited to active RFID tags, passive RFID tags, battery assisted passive RFID tags; or any other reasonable way to determine location. For ease, at times the above variations are not listed or are only partially listed; this is in no way meant to be a limitation.

As used herein, terms “cloud,” “Internet cloud,” “cloud computing,” “cloud architecture,” and similar terms correspond to at least one of the following: (1) a large number of computers connected through a real-time communication network (e.g., Internet); (2) providing the ability to run a program or application on many connected computers (e.g., physical machines, virtual machines (VMs)) at the same time; (3) network-based services, which appear to be provided by real server hardware, and are in fact served up by virtual hardware (e.g., virtual servers), simulated by software running on one or more real machines (e.g., allowing to be moved around and scaled up (or down) on the fly without affecting the end user).

In some embodiments, the illustrative computer-based systems or platforms of the present disclosure may be configured to securely store and/or transmit data by utilizing one or more of encryption techniques (e.g., private/public key pair, Triple Data Encryption Standard (3DES), block cipher algorithms (e.g., IDEA, RC2, RC5, CAST and Skipjack), cryptographic hash algorithms (e.g., MD5, RIPEMD-160, RTRO, SHA-1, SHA-2, Tiger (TTH), WHIRLPOOL, RNGs).

As used herein, the term “user” shall have a meaning of at least one user. In some embodiments, the terms “user”, “subscriber” “consumer” or “customer” should be understood to refer to a user of an application or applications as described herein and/or a consumer of data supplied by a data provider. By way of example, and not limitation, the terms “user” or “subscriber” can refer to a person who receives data provided by the data or service provider over the Internet in a browser session, or can refer to an automated software application which receives the data and stores or processes the data.

The aforementioned examples are, of course, illustrative and not restrictive.

At least some aspects of the present disclosure will now be described with reference to the following numbered clauses.

Publications cited throughout this document are hereby incorporated by reference in their entirety. While one or more embodiments of the present disclosure have been described, it is understood that these embodiments are illustrative only, and not restrictive, and that many modifications may become apparent to those of ordinary skill in the art, including that various embodiments of the inventive methodologies, the illustrative systems and platforms, and the illustrative devices described herein can be utilized in any combination with each other. Further still, the various steps may be carried out in any desired order (and any desired steps may be added and/or any desired steps may be eliminated).

1. A system comprising:
an incubation chamber that receives a filled well array;
wherein the filled well array comprises a well array having a sample of cells;
wherein the well array comprises at least one well;
an imaging device in or near the incubation chamber to capture a series of images of the filled well array during incubation in the incubation chamber;
wherein the imaging device communicates the series of images to at least one processor;
wherein the at least one processor is configured to:
detect each well in each image of the series of images of the filled well array, localize each well in each image of the series of images of the filled well array,
detect at least one cell of the sample of cells in each well of each image of the series of images of the filled well array,
track a relative location of each cell of the at least one cell in each well in each image of the series of images, and
determine cell-to-cell contact based at least in part on the relative location.

2. The system of claim 1, wherein the at least one processor is further configured to determine a number of cells in each well in each image of the series of images based at least in part on a prioritization of the at least one well.

3. The system of claim 1, further comprising at least one loader to receive the sample of cells and load the sample of cells into the well array to form a filled well array.

4. The system of claim 1, wherein the at least one processor is further configured to determine a prioritization of the at least one well based at least in part on based at least in part on the at least one cell in each well.

5. The system of claim 1, further comprising at least one applicator that dispenses at least one substance onto the sample of cells to prepare the sample of cells for imaging.

6. The system of claim 1, further comprising:
at least one vacuum chamber that receives the well array comprising at least one well; and
at least one plasma oxidizer in the at least one vacuum chamber to clean the well array in a vacuum.

7. The system of claim 1, further comprising at least one cell picker that receives the filled well array from the incubation chamber after incubation; and
wherein the at least one processor is configured to control the at least one cell picker to extract a set of cells from the filled well array based at least in part on a number of cells in each well in each image, relative location of each cell of the at least one cell in each well in each image, and the cell-to-cell contact.

8. A method comprising:
determining, by at least one processor, that a filled well array is positioned within an incubation chamber;
wherein the filled well array comprises a well array having a sample of cells;
wherein the well array comprises at least one well;
instructing, by the at least one processor, an imaging device in the incubation chamber to capture a series of images of the filled well array during incubation;
detecting, by at least one processor, each well in each image of the series of images of the filled well array,
localizing, by at least one processor, each well in each image of the series of images of the filled well array,
detecting, by at least one processor, at least one cell of the sample of cells in each well of each image of the series of images of the filled well array,
tracking, by at least one processor, a relative location of each cell of the at least one cell in each well in each image of the series of images, and
determining, by at least one processor, cell-to-cell contact based at least in part on the relative location.

9. The method of claim 8, further comprising determining, by the at least one processor, a number of cells in each well in each image of the series of images based at least in part on a prioritization of the at least one well.

10. The method of claim 8, further comprising instructing, by the at least one processor, at least one loader to receive the sample of cells and load the sample of cells into the well array to form a filled well array.

11. The method of claim 8, further comprising determining, by the at least one processor, a prioritization of the at least one well based at least in part on based at least in part on the at least one cell in each well.

12. The method of claim 8, further comprising instructing, by the at least one processor, at least one applicator to dispense at least one substance onto the sample of cells to prepare the sample of cells for imaging.

13. The method of claim 8, further comprising:
instructing, by the at least one processor, at least one vacuum chamber to receive the well array comprising at least one well; and
instructing, by the at least one processor, at least one plasma oxidizer in the at least one vacuum chamber to clean the well array in a vacuum.

14. The method of claim 8, further comprising:
instructing, by the at least one processor, at least one cell picker to receive the filled well array from the incubation chamber after incubation; and
instructing, by the at least one processor, the at least one cell picker to extract a set of cells from the filled well array based at least in part on a number of cells in each well in each image, relative location of each cell of the at least one cell in each well in each image, and the cell-to-cell contact.

15. A non-transitory computer readable medium having software instructions stored thereon, the software instructions configured to cause at least one processor to perform steps comprising:
determining that a filled well array is positioned within an incubation chamber;
wherein the filled well array comprises a well array having a sample of cells;
wherein the well array comprises at least one well;
instructing an imaging device in the incubation chamber to capture a series of images of the filled well array during incubation;
detecting each well in each image of the series of images of the filled well array, localizing each well in each image of the series of images of the filled well array,
detecting at least one cell of the sample of cells in each well of each image of the series of images of the filled well array,
tracking a relative location of each cell of the at least one cell in each well in each image of the series of images, and
determining cell-to-cell contact based at least in part on the relative location.

16. The non-transitory computer readable medium of claim 15, further comprising software instructions configured to cause the at least one processor to perform steps comprising determining a number of cells in each well in each image of the series of images based at least in part on a prioritization of the at least one well.

17. The non-transitory computer readable medium of claim 15, further comprising software instructions configured to cause the at least one processor to perform steps comprising:
instructing at least one loader to receive the sample of cells and load the sample of cells into the well array to form a filled well array; and
determining a prioritization of the at least one well based at least in part on based at least In part on the each well.

18. (canceled)

19. The non-transitory computer readable medium of claim 15, further comprising software instructions configured to cause the at least one processor to perform steps comprising instructing at least one applicator to dispense at least one substance onto the sample of cells to prepare the sample of cells for imaging.

20. The non-transitory computer readable medium of claim 15, further comprising software instructions configured to cause the at least one processor to perform steps comprising:
instructing at least one vacuum chamber to receive the well array comprising at least one well; and
instructing at least one plasma oxidizer in the at least one vacuum chamber to clean the well array in a vacuum.

21. The non-transitory computer readable medium of claim 15, further comprising software instructions configured to cause the at least one processor to perform steps comprising:
instructing at least one cell picker to receive the filled well array from the incubation chamber after incubation; and
instructing the at least one cell picker to extract a set of cells from the filled well array based at least in part on a number of cells in each well in each image, relative location of each cell of the at least one cell in each well in each image, and the cell-to-cell contact.
